(19) United States
(12) Patent Application Publication
(10) Pub. No.: US 2009/0092660 A1
(43) Pub. Date: Apr. 9, 2009

(54) Methods and compositions for needleless delivery of particles

(75) Inventor: Randall J. Mrsny, Los Altos, CA (US)

Correspondence Address: JONES DAY, 222 EAST 41ST ST, NEW YORK, NY, 10017, US

(73) Assignee: Trinity Biosystems, Inc., Menlo Park, CA (US)

(21) Appl. No.: 11/891,475

(22) Filed: Aug. 8, 2007

Related U.S. Application Data
(60) Provisional application No. 60/836,855, filed on Aug. 9, 2006.

(51) Int. Cl. A61K 9/127; A61K 9/14; A61K 45/00; A61K 9/50; A61P 43/00

(52) U.S. Cl. 424/450; 424/489; 424/93.7; 424/499

(57) ABSTRACT

Methods and compositions for needleless delivery of particles to the bloodstream of a subject are provided herein. In one aspect, the invention provides a delivery construct, comprising a receptor binding domain, a transcytosis domain, a particle to be delivered to a subject, and, optionally, a cleavable linker. In other aspects, the invention provides compositions comprising delivery constructs of the invention, kits comprising delivery constructs of the invention, and methods of using delivery constructs of the invention.

### 1. CROSS-REFERENCE TO RELATED APPLICATION

This application is entitled to and claims benefit of U.S. Provisional Application No. 60/836,855, filed Aug. 9, 2006, which is hereby incorporated by reference in its entirety.

### 2. FIELD OF THE INVENTION

The present invention relates, in part, to methods and compositions for needleless delivery of particles to a subject. In one aspect, the methods and compositions involve administering to the subject a delivery construct comprising the particle to be delivered.

### 3. BACKGROUND

Particles have been used in modern medicine in a variety of different applications. For example, gold and platinum particles have been used in cancer and arthritis therapies; high-contrast particles have been used in imaging applications; lipospheres and porous particles have been used in drug-delivery applications; and whole cells have been administered in certain experimental cancer therapies. Administration of these particles can result in drastic improvements in quality of life for subjects afflicted with a wide range of ailments.

However, administration of these particles remains problematic. Currently, particles are typically administered by injection. Such injections require penetration of the subject's skin and tissues and are associated with pain. Further, penetration of the skin breaches one effective nonspecific mechanism of protection against infection, and thus can lead to potentially serious infection.

Accordingly, there is an unmet need for new methods and compositions that can be used to administer particles to subjects without breaching the skin of the subject. This and other needs are met by the methods and compositions of the present invention.

### 4. SUMMARY OF THE INVENTION

The delivery constructs of the invention comprise a particle for delivery to a subject. Accordingly, in certain aspects, the invention provides a delivery construct comprising a receptor binding domain, a transcytosis domain, and a particle to be delivered to a subject. Optionally, the particle can be connected to the remainder of the delivery construct with a cleavable linker. In such embodiments, cleavage at the cleavable linker can separate the particle from the remainder of the delivery construct.

The particle can be any particle that is desired to be introduced into a subject. Thus, the particle can be, for example, a metal, a liposphere, a porous particle, a cell (either living or dead), a high-contrast particle, a coated particle, a peptide or polypeptide aggregate, a peptide or polypeptide crystal, or any combination thereof. In certain embodiments, the particle is a liposphere. In certain embodiments, the particle is a porous particle. In certain embodiments, the particle is a cell. In certain embodiments, the particle is a high-contrast particle. In certain embodiments, the particle is a peptide or polypeptide aggregate. In certain embodiments, the particle is a peptide or polypeptide crystal.

In yet another aspect, the invention provides a composition comprising a delivery construct of the invention. In certain embodiments, the composition is a pharmaceutical composition.

In still another aspect, the invention provides a method for delivering a particle to a subject. The method comprises contacting an apical surface of a polarized epithelial cell of the subject with a delivery construct of the invention. The delivery construct can comprise a receptor binding domain, a transcytosis domain, the particle to be delivered, and, optionally, a cleavable linker. The transcytosis domain can transcytose the particle to and through the basal-lateral membrane of the epithelial cell. In certain embodiments, the cleavable linker can be cleaved by an enzyme that is present at a basal-lateral membrane of a polarized epithelial cell of the subject. In other embodiments, the cleavable linker can be cleavable by an enzyme that is present in the plasma of the subject. Cleavage at the cleavable linker can separate the particle from the remainder of the delivery construct, and can deliver the particle to the subject free from the remainder of the construct.

### 5. BRIEF DESCRIPTION OF THE DRAWINGS

FIG. 1 presents the amino acid sequence of an exemplary PE.

FIGS. 2A-F present confocal micrographs showing co-localization of ntPE-GFP and particles following coupling of ntPE-GFP to the particles (FIGS. 2A-C), while BSA coupled to similar particles does not exhibit GFP activity (FIGS. 2D-F).

FIG. 3 presents a diagram showing various coupling orientations of ntPE-GFP to an exemplary particle; the receptor binding domain is labeled as domain 1, the transcytosis domain is labeled as domain 2, and the GFP domain is unlabeled.

FIGS. 4A-4C present photographs comparing transport of ntPE-GFP (FIG. 4A), K57 ntPE-GFP (FIG. 4B), and GFP (FIG. 4C) conjugated to particles across confluent monolayers of polarized Caco-2 epithelial cells. The photographs presented in FIGS. 4A-C were taken about six hours following application of the three particle conjugates to the apical surface of the polarized monolayer.

FIG. 5 presents a photograph showing transport of ntPE-GFP conjugated to particles across confluent monolayers of polarized Caco-2 epithelial cells. The photograph presented in FIG. 5 was taken about 24 hours following application of the particle conjugate to the apical surface of the polarized monolayer.

FIG. 6 presents a graphical representation of serum glucose concentrations following administration of insulin aggregates conjugated to ntPE or PBS to female BALB/c mice. Administration is either by oral gavage or subcutaneous injection, and two different exemplary conjugates were tested to assess the effect of the ratio of ntPE to particle on delivery.

### 6. DETAILED DESCRIPTION OF THE INVENTION

### 6.1. Definitions

Unless defined otherwise, all technical and scientific terms used herein have the meaning commonly understood by a person skilled in the art to which this invention belongs. As used herein, the following terms have the meanings ascribed to them unless specified otherwise.

A “ligand” is a compound that specifically binds to a target molecule. Exemplary ligands include, but are not limited to, an antibody, a cytokine, a substrate, a signaling molecule, and the like.

A “receptor” is a compound that specifically binds to a ligand.

A ligand or a receptor (e.g., an antibody) “specifically binds to” or “is specifically immunoreactive with” another molecule when the ligand or receptor functions in a binding reaction that indicates the presence of the molecule in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to another polynucleotide comprising a complementary sequence and an antibody specifically binds under immunoassay conditions to an antigen bearing an epitope used to induce the antibody.

“Immunoassay” refers to a method of detecting an analyte in a sample involving contacting the sample with an antibody that specifically binds to the analyte and detecting binding between the antibody and the analyte. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988) Antibodies, A Laboratory Manual, Cold Spring Harbor Publications, New York, for a description of immunoassay formats and conditions that can be used to determine specific immunoreactivity. In one example, an antibody that binds a particular antigen with an affinity (Kₘ) of about 10 μM specifically binds the antigen.

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences. A “cleavable linker” refers to a linker that can be degraded or otherwise severed to separate the two components connected by the cleavable linker. Cleavable linkers are generally cleaved by enzymes, typically peptidases, proteases, nucleases, lipases, and the like. Cleavable linkers may also be cleaved by environmental cues, such as, for example, changes in temperature, pH, salt concentration, etc. when there is such a change in environment following transcytosis of the delivery construct across a polarized epithelial membrane.

A “particle,” according to the present invention, refers to a homogenous or heterogenous particle that is from about 10 nm to about 150 nm in diameter. A particle can be of regular or irregular shape, and can be perfectly or roughly spherical, square, or any other shape known to one of skill in the art without limitation. Exemplary particles include, but are not limited to, metal particles, liposheres, polymeric particles, high-contrast particles such as those used in imagining applications, and the like.

A “pharmaceutical composition” refers to a composition suitable for pharmaceutical use in an animal. A pharmaceutical composition comprises a pharmacologically effective amount of an active agent and a pharmaceutically acceptable carrier. “Pharmacologically effective amount” refers to that amount of an agent effective to produce the intended pharmacological result. “Pharmaceutically acceptable carrier” refers to any of the standard pharmaceutical carriers, vehicles, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 21st Ed. 2005, Mack Publishing Co., Easton. A “pharmaceutically acceptable salt” is a salt that can be formulated into a compound for pharmaceutical use including, e.g., metal salts (sodium, potassium, magnesium, calcium, etc.) and salts of ammonia or organic amines.

Preferred pharmaceutical carriers depend upon the intended mode of administration of the active agent. Typical modes of administration include enteral (e.g., oral, intranasal, rectal, or vaginal) or parenteral (e.g., subcutaneous, intramuscular, intravenous or intraperitoneal injection; or topical (e.g., transdermal, or transmucosal administration).

A “small organic molecule” refers to organic molecules of a size comparable to those organic molecules generally used in pharmaceuticals. The term excludes organic biopolymers (e.g., proteins, nucleic acids, etc.). Preferred small organic molecules range in size up to about 5000 Da, up to about 2000 Da, or up to about 1000 Da.

A “subject” of diagnosis, treatment, or administration is a human or non-human animal, including a mammal or a primate, and preferably a human.

“Treatment” refers to prophylactic treatment or therapeutic treatment. A “prophylactic” treatment is a treatment administered to a subject who does not exhibit signs of a disease or exhibits only early signs for the purpose of decreasing the risk of developing pathology. A “therapeutic” treatment is a treatment administered to a subject who exhibits signs of pathology for the purpose of diminishing or eliminating those signs.

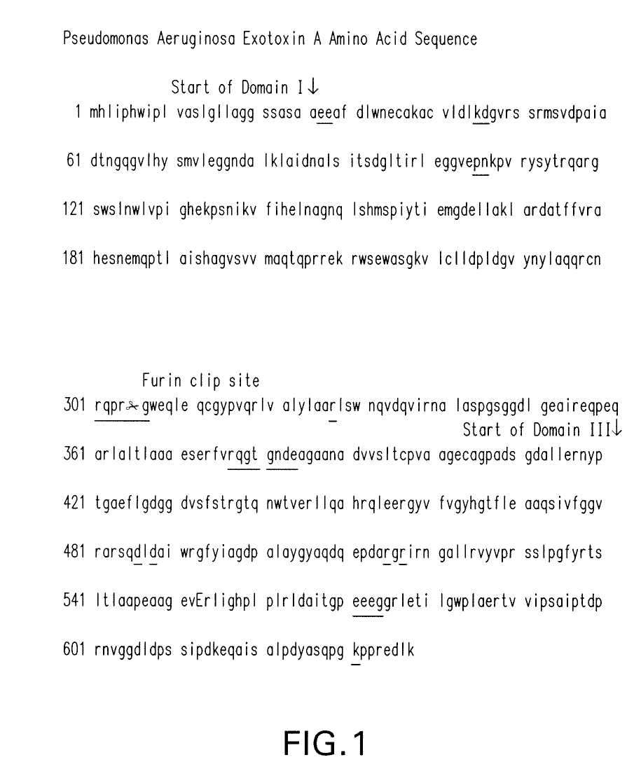

“Pseudomonas exotoxin A” or “PE” is secreted by Pseudomonas aeruginosa as a 67 kD protein composed of three prominent globular domains (Ia, II, and III) and one small subdomain (Ib) that connects domains II and III. See A. S. Allured et al., 1986, Proc. Natl. Acad. Sci. 83:1320-1324. Without intending to be bound to any particular theory or mechanism of action, domain Ia of PE is believed to mediate cell binding because domain Ia specifically binds to the low density lipoprotein receptor-related protein (“LRP”), also known as the α2-macroglobulin receptor (“α2-MR”) and CD-91. See M. Z. Kounnas et al., 1992, J. Biol. Chem. 267:12420-23. Domain Ia spans amino acids 1-252. Domain II of PE is believed to mediate transcytosis to the interior of a cell following binding of domain Ia to the α2-MR. Domain II spans amino acids 253-364. Certain portions of this domain may be required for secretion of PE from Pseudomonas aeruginosa after its synthesis. See, e.g., Vouloux et al., 2000, J. Bacterol. 182:4051-8. Domain Ib has no known function and spans amino acids 365-399. Domain III mediates cytotoxicity of PE and includes an endoplasmic reticulum retention sequence. PE cytotoxicity is believed to result from ADP ribosylation of elongation factor 2, which inactivates protein synthesis. Domain III spans amino acids 400-613 of PE. Deleting amino acid E553 (“ΔE553”) from domain III eliminates EF2 ADP ribosylation activity and detoxifies PE. PE having the mutation ΔE553 is referred to herein as “PEΔE553.” Genetically modified forms of PE are described in, e.g., U.S. Pat. Nos. 5,602,095; 5,512,658 and 5,458,878. Pseudomonas exotoxin, as used herein, also includes genetically modified, allelic, and chemically inactivated forms of PE within this definition. See, e.g., Vasil et al., 1986, Infect. Immunol. 52:538-48. Further, reference to the various domains of PE is made herein to the reference PE sequence presented as FIG. 1. However, one or more domain from modified PE, e.g., genetically or chemically modified PE, or a portion of such domains, can also be used in the chimeric immunogens of the invention so long as the domains retain functional activity. One of skill in the art can readily identify such domains of such modified PE based on, for example, homology to the PE amino acid sequence exemplified in FIG. 1 and test for functional activity using, for example, the assays described below.

“Polynucleotide” refers to a polymer composed of nucleotide units. Polynucleotides include naturally occurring nucleic acids, such as deoxyribonucleic acid (“DNA”) and ribonucleic acid (“RNA”) as well as nucleic acid analogs. Nucleic acid analogs include those which include non-naturally occurring bases, nucleotides that engage in linkages with other nucleotides other than the naturally occurring phosphodiester bond or which include bases attached through linkages other than phosphodiester bonds. Thus, nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like. Such polynucleotides can be synthesized, for example, using an automated DNA synthesizer. The term “nucleic acid” typically refers to large polynucleotides. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

“Complementary” refers to the topological compatibility or matching together of interacting surfaces of two polynucleotides. Thus, the two molecules can be described as complementary, and furthermore, the contact surface characteristics are complementary to each other. A first polynucleotide is complementary to a second polynucleotide if the nucleotide sequence of the first polynucleotide is substantially identical to the nucleotide sequence of the polynucleotide binding partner of the second polynucleotide, or if the first polynucleotide can hybridize to the second polynucleotide under stringent hybridization conditions. Thus, the polynucleotide whose sequence is 5′-TATAC-3′ is complementary to a polynucleotide whose sequence is 5′-GTATA-3′.

The term “% sequence identity” is used interchangeably herein with the term “% identity” and refers to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm, and means that a given sequence is at least 80% identical to another length of another sequence. Exemplary levels of sequence identity include, but are not limited to, 60%, 70%, 80%, 85%, 90%, 95%, 98% or greater sequence identity to a given sequence.

The term “% sequence homology” is used interchangeably herein with the term “% homology” and refers to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence. Exemplary levels of sequence homology include, but are not limited to, 60%, 70%, 80%, 85%, 90%, 95%, 98% or greater sequence homology to a given sequence.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at the NCBI website. See also Altschul et al., 1990, J. Mol. Biol. 215:403-10 (with special reference to the published default setting, i.e., parameters w=4, t=17) and Altschul et al., 1997, Nucleic Acids Res., 25:3389-3402. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix. See id.

A preferred alignment of selected sequences in order to determine “% identity” between two or more sequences, is performed using for example, the CLUSTAL-W program in MacVector version 6.5, operated with default parameters, including an open gap penalty of 10.0, an extended gap penalty of 0.1, and a BLOSUM 30 similarity matrix.

“Polar Amino Acid” refers to a hydrophilic amino acid having a side chain that is uncharged at physiological pH, but which has at least one bond in which the pair of electrons shared in common by two atoms is held more closely by one of the atoms. Genetically encoded polar amino acids include Asn (N), Gln (Q) Ser (S) and Thr (T).

“Nonpolar Amino Acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded nonpolar amino acids include Ala (A), Gly (G), Ile (I), Leu (L), Met (M) and Val (V).

“Hydrophilic Amino Acid” refers to an amino acid exhibiting a hydrophobicity of less than zero according to the normalized consensus hydrophobicity scale of Eisenberg et al., 1984, J. Mol. Biol. 179:125-142. Genetically encoded hydrophilic amino acids include Arg (R), Asn (N), Asp (D), Glu (E), Gln (O), H is (H), Lys (K), Ser (S) and Thr (T).

“Hydrophobic Amino Acid” refers to an amino acid exhibiting a hydrophobicity of greater than zero according to the normalized consensus hydrophobicity scale of Eisenberg et al., 1984, J. Mol. Biol. 179:125-142. Genetically encoded hydrophobic amino acids include Ala (A), Gly (G), Ile (I), Leu (L), Met (M), Phe (F), Pro (P), Trp (W), Tyr (Y) and Val (V).

“Acidic Amino Acid” refers to a hydrophilic amino acid having a side chain pK value of less than 7. Acidic amino acids typically have negatively charged side chains at physiological pH due to loss of a hydrogen ion. Genetically encoded acidic amino acids include Asp (D) and Glu (E).

“Basic Amino Acid” refers to a hydrophilic amino acid having a side chain pK value of greater than 7. Basic amino acids typically have positively charged side chains at physiological pH due to association with a hydrogen ion. Genetically encoded basic amino acids include Arg (R), His (H) and Lys (K).

“Encoding” refers to the inherent property of specific sequences of nucleotides in a polynucleotide, such as a gene, a cDNA, or an mRNA, to serve as templates for synthesis of other polymers and particles in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Amplification” refers to any means by which a polynucleotide sequence is copied and thus expanded into a larger number of polynucleotide molecules, e.g., by reverse transcription, polymerase chain reaction, ligase chain reaction, and the like.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

“Probe,” when used in reference to a polynucleotide, refers to a polynucleotide that is capable of specifically hybridizing to a designated sequence of another polynucleotide. A probe specifically hybridizes to a target complementary polynucleotide, but need not reflect the exact complementary sequence of the template. In such a case, specific hybridization of the probe to the target depends on the stringency of the hybridization conditions. Probes can be labeled with, e.g., chromogenic, radioactive, or fluorescent moieties and used as detectable moieties. In instances where a probe provides a point of initiation for synthesis of a complementary polynucleotide, a probe can also be a primer.

“Hybridizing specifically to” or “specific hybridization” or “selectively hybridize to” refers to the binding, duplexing, or hybridizing of a nucleic acid molecule preferentially to a particular nucleotide sequence under stringent conditions. The sequence can be present in a complex mixture (e.g., total cellular) of DNA or RNA or a combination thereof.

The term “stringent conditions” refers to conditions under which a probe will hybridize preferentially to its target subsequence, and to a lesser extent to, or not at all to, other sequences. “Stringent hybridization” and “stringent hybridization wash conditions” in the context of nucleic acid hybridization experiments such as Southern and northern hybridizations are sequence dependent, and are different under different environmental parameters. An extensive guide to the hybridization of nucleic acids can be found in Tijssen, 1993, Laboratory Techniques in Biochemistry and Molecular Biology—Hybridization with Nucleic Acid Probes, part I, chapter 2, “Overview of principles of hybridization and the strategy of nucleic acid probe assays”, Elsevier, N.Y.; Sambrook et al., 2001, Molecular Cloning: A Laboratory Manual, Cold Spring Harbor Laboratory, 3^{rd} ed., NY; and Ausubel et al., eds., Current Edition, Current Protocols in Molecular Biology, Greene Publishing Associates and Wiley Interscience, NY.

Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe.

One example of stringent hybridization conditions for hybridization of complementary nucleic acids which have more than about 100 complementary residues on a filter in a Southern or northern blot is 50% formalin with 1 mg of heparin at 42° C., with the hybridization being carried out overnight. An example of highly stringent wash conditions is 0.15 M NaCl at 72° C. for about 15 minutes. An example of stringent wash conditions is a 0.2×SSC wash at 65° C. for 15 minutes. See Sambrook et al. for a description of SSC buffer. A high stringency wash can be preceded by a low stringency wash to remove background probe signal. An exemplary medium stringency wash for a duplex of, e.g., more than about 100 nucleotides, is 1×SSC at 45° C. for 15 minutes. An exemplary low stringency wash for a duplex of, e.g., more than about 100 nucleotides, is 4-6×SSC at 40° C. for 15 minutes. In general, a signal to noise ratio of 2× (or higher) than that observed for an unrelated probe in the particular hybridization assay indicates detection of a specific hybridization.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer. Conventional notation is used herein to portray polypeptide sequences; the beginning of a polypeptide sequence is the amino-terminus, while the end of a polypeptide sequence is the carboxyl-terminus.

The term “protein” typically refers to large polypeptides, for example, polypeptides comprising more than about 50 amino acids. The term “protein” can also refer to dimers, trimers, and multimers that comprise more than one polypeptide.

“Conservative substitution” refers to the substitution in a polypeptide of an amino acid with a functionally similar amino acid. The following six groups each contain amino acids that are conservative substitutions for one another:
Alanine (A), Serine (S), and Threonine (T)
Aspartic acid (D) and Glutamic acid (E)
Asparagine (N) and Glutamine (Q)
Arginine (R) and Lysine (K)
Isoleucine (I), Leucine (L), Methionine (M), and Valine (V)
Phenylalanine (F), Tyrosine (Y), and Tryptophan (W).

The term “about,” as used herein, unless otherwise indicated, refers to a value that is no more than 10% above or below the value being modified by the term. For example, the term “about 5 μg/kg” means a range of from 4.5 μg/kg to 5.5 μg/kg. As another example, “about 1 hour” means a range of from 48 minutes to 72 minutes.

### 6.2. Delivery Constructs

Generally, the delivery constructs of the present invention comprise polypeptides that comprise structural domains corresponding to domains Ia and II of PE. These structural domains perform certain functions, including, but not limited to, cell recognition and transcytosis, that correspond to the functions of the domains of PE.

In addition to the portions of the molecule that correspond to PE functional domains, the delivery constructs of this invention further comprise a particle for delivery to a biological compartment of a subject. The particle can be introduced into or connected with any portion of the delivery construct that does not disrupt a cell-binding or transcytosis activity. Optionally, the particle can be connected with the remainder of the delivery construct with a cleavable linker.

Accordingly, the delivery constructs of the invention generally comprise the following structural elements, each element imparting particular functions to the delivery construct: (1) a “receptor binding domain” that functions as a ligand for a cell surface receptor and that mediates binding of the construct to a cell; (2) a “transcytosis domain” that mediates transcytosis from a lumen bordering the apical surface of a mucous membrane to the basal-lateral side of a mucous membrane; and (3) the particle. Optionally, the delivery constructs can also comprise a cleavable linker that connects the particle to the remainder of the delivery construct.

The delivery constructs of the invention offer several advantages over conventional techniques for local or systemic delivery of particles to a subject. Foremost among such advantages is the ability to deliver the particle without using a needle to puncture the skin of the subject. Many subjects require repeated, regular doses of particles. For example, subjects must be repeatedly injected with platinum-based cancer therapeutics during the course of such therapies. Such subjects' quality of life would be greatly improved if the delivery of a particle could be accomplished without injection, by avoiding pain or potential complications associated therewith.

In addition, embodiments where the particle is connected with the remainder of the delivery construct with a cleavable linker allows the particle to be liberated from the delivery construct and released from the remainder of the delivery construct after transcytosis across the epithelial membrane. Such liberation reduces the probability of induction of an immune response against the particle. It also allows the particle to interact with its target free from the remainder of the delivery construct.

Other advantages of the delivery constructs of the invention will be apparent to those of skill in the art.

Accordingly, in certain embodiments, the invention provides a delivery construct that comprises a receptor binding domain, a transcytosis domain, a particle to be delivered to a subject. Optionally, the particle can be connected with the remainder of the delivery construct with a cleavable linker. Cleavage at the optional cleavable linker can separate the particle from the remainder of the construct. The cleavable linker can be, for example, cleavable by an enzyme that is present at a basal-lateral membrane of a polarized epithelial cell of the subject or in the plasma of the subject.

In certain embodiments, the particle is a metal particle, a liposphere, a porous particle, a cell, a peptide or polypeptide aggregate, a peptide or polypeptide crystal, or a high-contrast particle.

In certain embodiments, the delivery construct further comprises a second cleavable linker. In certain embodiments, the first and/or the second cleavable linker comprises an amino acid sequence that is selected from the group consisting of Ala-Ala-Pro-Phe (SEQ ID NO.:4), Gly-Gly-Phe (SEQ ID NO.:5), Ala-Ala-Pro-Val (SEQ ID NO.:6), Gly-Gly-Leu (SEQ ID NO.:7), Ala-Ala-Leu (SEQ ID NO.:8), Phe-Val-Arg (SEQ ID NO.:9), Val-Gly-Arg (SEQ ID NO.:10). In certain embodiments, the first and/or the second cleavable linker comprises an amino acid sequence that is selected from the group consisting of Ala-Ala-Pro-Phe (SEQ ID NO.:4), Gly-Gly-Phe (SEQ ID NO.:5), Ala-Ala-Pro-Val (SEQ ID NO.:6), Gly-Gly-Leu (SEQ ID NO.:7), Ala-Ala-Leu (SEQ ID NO.:8), Phe-Val-Arg (SEQ ID NO.:9), Val-Gly-Arg (SEQ ID NO.:10) and is cleavable by an enzyme that exhibits higher activity on the basal-lateral side of a polarized epithelial cell than it does on the apical side of the polarized epithelial cell. In certain embodiments, the first and/or the second cleavable linker comprises an amino acid sequence that is selected from the group consisting of Ala-Ala-Pro-Phe (SEQ ID NO.:4), Gly-Gly-Phe (SEQ ID NO.:5), Ala-Ala-Pro-Val (SEQ ID NO.:6), Gly-Gly-Leu (SEQ ID NO.:7), Ala-Ala-Leu (SEQ ID NO.:8), Phe-Val-Arg (SEQ ID NO.:9), Val-Gly-Arg (SEQ ID NO.:10) and is cleavable by an enzyme that exhibits higher activity in the plasma than it does on the apical side of a polarized epithelial cell.

In certain embodiments, the enzyme that is present at a basal-lateral membrane of a polarized epithelial cell is selected from the group consisting of Cathepsin GI, Chymotrypsin I, Elastase I, Subtilisin AI, Subtilisin AII, Thrombin I, and Urokinase I.

In certain embodiments, the receptor binding domain is selected from the group consisting of receptor binding domains from Pseudomonas exotoxin A, cholera toxin, botulinum toxin, diphtheria toxin, shiga toxin, or shiga-like toxin; monoclonal antibodies; polyclonal antibodies; single-chain antibodies; TGF α; EGF; IGF-I; IGF-II; IGF-III; IL-1; IL-2; IL-3; IL-6; MIP-1a; MIP-1b; MCAF; and IL-8. In certain embodiments, the receptor binding domain binds to a cell-surface receptor that is selected from the group consisting of α2-macroglobulin receptor, epidermal growth factor receptor, transferrin receptor, chemokine receptor, CD25, CD11B, CD11C, CD80, CD86, TNFα receptor, TOLL receptor, M-CSF receptor, GM-CSF receptor, scavenger receptor, and VEGF receptor. In further embodiments, the receptor binding domain of Pseudomonas exotoxin A is Domain Ia of Pseudomonas exotoxin A. In yet further embodiments, the receptor binding domain of Pseudomonas exotoxin A has an amino acid sequence that is SEQ ID NO.:1.

In certain embodiments, the transcytosis domain is selected from the group consisting of transcytosis domains from Pseudomonas exotoxin A, botulinum toxin, diphtheria toxin, pertussis toxin, cholera toxin, heat-labile E. coli enterotoxin, shiga toxin, and shiga-like toxin. In further embodiments, the transcytosis domain is Pseudomonas exotoxin A transcytosis domain. In still further embodiments, the Pseudomonas exotoxin A transcytosis domain has an amino acid sequence that is SEQ ID NO.:2.

### 6.2.1. Receptor Binding Domain

The delivery constructs of the invention generally comprise a receptor binding domain. The receptor binding domain can be any receptor binding domain known to one of skill in the art, without limitation, to bind to a cell surface receptor that is present on the apical membrane of an epithelial cell. Preferably, the receptor binding domain binds specifically to the cell surface receptor. The receptor binding domain should bind to the cell surface receptor with sufficient affinity to allow endocytosis of the delivery construct.

In certain embodiments, the receptor binding domain can comprise a peptide, a polypeptide, a protein, a lipid, a carbohydrate, or a small organic molecule, or a combination thereof. Examples of each of these molecules that bind to cell surface receptors present on the apical membrane of epithelial cells are well known to those of skill in the art. Suitable peptides or polypeptides include, but are not limited to, bacterial toxin receptor binding domains, such as the receptor binding domains from PE, cholera toxin, botulinum toxin, diphtheria toxin, shiga toxin, shiga-like toxin, etc.; antibodies, including monoclonal, polyclonal, and single-chain antibodies, or derivatives thereof, growth factors, such as EGF, IGF-I, IGF-II, IGF-III etc.; cytokines, such as IL-1, IL-2, IL-3, IL-6, etc; chemokines, such as MIP-1a, MIP-1b, MCAF, IL-8, etc.; and other ligands, such as CD4, cell adhesion molecules from the immunoglobulin superfamily, integrins, ligands specific for the IgA receptor, etc. See, e.g., Pastan et al., 1992, Annu. Rev. Biochem. 61:331-54; and U.S. Pat. Nos. 5,668,255, 5,696,237, 5,863,745, 5,965,406, 6,022,950, 6,051,405, 6,251,392, 6,440,419, and 6,488,926. The skilled artisan can select the appropriate receptor binding domain based upon the expression pattern of the receptor to which the receptor binding domain binds.

Lipids suitable for receptor binding domains include, but are not limited to, lipids that themselves bind cell surface receptors, such as sphingosine-1-phosphate, lysophosphatidic acid, sphingosylphosphorylcholine, retinoic acid, etc.; lipoproteins such as apolipoprotein E, apolipoprotein A, etc., and glycolipids such as lipopolysaccharide, etc.; glycosphingolipids such as globotriaosylceramide and galabiosylceramide; and the like. Carbohydrates suitable for receptor binding domains include, but are not limited to, monosaccharides, disaccharides, and polysaccharides that comprise simple sugars such as glucose, fructose, galactose, etc.; and glycoproteins such as mucins, selectins, and the like. Suitable small organic molecules for receptor binding domains include, but are not limited to, vitamins, such as vitamin A, B₁, B₂, B₃, B₆, B₉, B₁₂, C, D, E, and K, amino acids, and other small molecules that are recognized and/or taken up by receptors present on the apical surface of epithelial cells. U.S. Pat. No. 5,807,832 provides an example of such small organic molecule receptor binding domains, vitamin B₁₂.

In certain embodiments, the receptor binding domain can bind to a receptor found on an epithelial cell. In further embodiments, the receptor binding domain can bind to a receptor found on the apical membrane of an epithelial cell. The receptor binding domain can bind to any receptor known to be present on the apical membrane of an epithelial cell by one of skill in the art without limitation. For example, the receptor binding domain can bind to α2-MR, EGFR, or IGFR. An example of a receptor binding domain that can bind to α2-MR is domain Ia of PE. Accordingly, in certain embodiments, the receptor binding domain is domain Ia of PE. In other embodiments, the receptor binding domain is a portion of domain Ia of PE that can bind to α2-MR. Exemplary receptor binding domains that can bind to EGFR include, but are not limited to, EGF and TGFα. Examples of receptor binding domains that can bind to IGFR include, but are not limited to, IGF-I, IGF-II, or IGF-III. Thus, in certain embodiments, the receptor binding domain is EGF, IGF-I, IGF-II, or IGF-III. In other embodiments, the receptor binding domain is a portion of EGF, IGF-I, IGF-II, or IGF-III that can bind to the EGF or IGF receptor.

In certain embodiments, the receptor binding domain binds to a receptor that is highly expressed on the apical membrane of a polarized epithelial cell but is not expressed or expressed at low levels on antigen presenting cells, such as, for example, dendritic cells. Exemplary receptor binding domains that have this kind of expression pattern include, but are not limited to, TGFα, EGF, IGF-I, IGF-II, and IGF-III.

In certain embodiments, the delivery constructs of the invention comprise more than one domain that can function as a receptor binding domain. For example, the delivery construct can comprise PE domain Ia in addition to another receptor binding domain.

The receptor binding domain can be attached to the remainder of the delivery construct by any method or means known by one of skill in the art to be useful for attaching such molecules, without limitation. In certain embodiments, the receptor binding domain is expressed together with the remainder of the delivery construct as a fusion protein. Such embodiments are particularly useful when the receptor binding domain and the remainder of the construct are formed from peptides or polypeptides.

In other embodiments, the receptor binding domain is connected with the remainder of the delivery construct with a linker. In yet other embodiments, the receptor binding domain is connected with the remainder of the delivery construct without a linker. Either of these embodiments are useful when the receptor binding domain comprises a peptide, polypeptide, protein, lipid, carbohydrate, nucleic acid, or small organic molecule.

In certain embodiments, the linker can form a covalent bond between the receptor binding domain and the remainder of the delivery construct. In certain embodiments, the covalent bond can be a peptide bond. In other embodiments, the linker can link the receptor binding domain to the remainder of the delivery construct with one or more non-covalent interactions of sufficient affinity. One of skill in the art can readily recognize linkers that interact with each other with sufficient affinity to be useful in the delivery constructs of the invention. For example, biotin can be attached to the receptor binding domain, and streptavidin can be attached to the remainder of the molecule. In certain embodiments, the linker can directly link the receptor binding domain to the remainder of the molecule. In other embodiments, the linker itself comprises two or more molecules that associate in order to link the receptor binding domain to the remainder of the molecule. Exemplary linkers include, but are not limited to, straight or branched-chain carbon linkers, heterocyclic carbon linkers, substituted carbon linkers, unsaturated carbon linkers, aromatic carbon linkers, peptide linkers, etc.

In embodiments where a linker is used to connect the receptor binding domain to the remainder of the delivery construct, the linkers can be attached to the receptor binding domain and/or the remainder of the delivery construct by any means or method known by one of skill in the art without limitation. For example, the linker can be attached to the receptor binding domain and/or the remainder of the delivery construct with an ether, ester, thioether, thioester, amide, imide, disulfide, peptide, or other suitable moiety. The skilled artisan can select the appropriate linker and method for attaching the linker based on the physical and chemical properties of the chosen receptor binding domain and the linker. The linker can be attached to any suitable functional group on the receptor binding domain or the remainder of the molecule. For example, the linker can be attached to sulfhydryl (—S), carboxylic acid (COOH) or free amine (—NH2) groups, which are available for reaction with a suitable functional group on a linker. These groups can also be used to connect the receptor binding domain directly connected with the remainder of the molecule in the absence of a linker.

Further, the receptor binding domain and/or the remainder of the delivery construct can be derivatized in order to facilitate attachment of a linker to these moieties. For example, such derivatization can be accomplished by attaching suitable derivative such as those available from Pierce Chemical Company, Rockford, Ill. Alternatively, derivatization may involve chemical treatment of the receptor binding domain and/or the remainder of the molecule. For example, glycol cleavage of the sugar moiety of a carbohydrate or glycoprotein receptor binding domain with periodate generates free aldehyde groups. These free aldehyde groups may be reacted with free amine or hydrazine groups on the remainder of the molecule in order to connect these portions of the molecule. See, e.g., U.S. Pat. No. 4,671,958. Further, the skilled artisan can generate free sulfhydryl groups on proteins to provide a reactive moiety for making a disulfide, thioether, thioester, etc. linkage. See, e.g., U.S. Pat. No. 4,659,839.

Any of these methods for attaching a linker to a receptor binding domain and/or the remainder of a delivery construct can also be used to connect a receptor binding domain with the remainder of the delivery construct in the absence of a linker. In such embodiments, the receptor binding domain is coupled with the remainder of the construct using a method suitable for the particular receptor binding domain. Thus, any method suitable for connecting a protein, peptide, polypeptide, nucleic acid, carbohydrate, lipid, or small organic molecule to the remainder of the delivery construct known to one of skill in the art, without limitation, can be used to connect the receptor binding domain to the remainder of the construct. In addition to the methods for attaching a linker to a receptor binding domain or the remainder of a delivery construct, as described above, the receptor binding domain can be connected with the remainder of the construct as described, for example, in U.S. Pat. Nos. 6,673,905; 6,585,973; 6,596,475; 5,856,090; 5,663,312; 5,391,723; 6,171,614; 5,366,958; and 5,614,503.

In certain embodiments, the receptor binding domain can be a monoclonal antibody. In some of these embodiments, the chimeric immunogen is expressed as a fusion protein that comprises an immunoglobulin heavy chain from an immunoglobulin specific for a receptor on a cell to which the chimeric immunogen is intended to bind. The light chain of the immunoglobulin then can be co-expressed with the chimeric immunogen, thereby forming a light chain-heavy chain dimer. In other embodiments, the antibody can be expressed and assembled separately from the remainder of the chimeric immunogen and chemically linked thereto.

### 6.2.2. Transcytosis Domain

The delivery constructs of the invention also comprise a transcytosis domain. The transcytosis domain can be any transcytosis domain known by one of skill in the art to effect transcytosis of chimeric proteins that have bound to a cell surface receptor present on the apical membrane of an epithelial cell. In certain embodiments, the transcytosis domain is a transcytosis domain from PE, diphtheria toxin, pertussis toxin, cholera toxin, heat-labile E. coli enterotoxin, shiga toxin, or shiga-like toxin. See, for example, U.S. Pat. Nos. 5,965,406, and 6,022,950. In preferred embodiments, the transcytosis domain is domain II of PE.

The transcytosis domain need not, though it may, comprise the entire amino acid sequence of domain II of native PE, which spans residues 253-364 of PE. For example, the transcytosis domain can comprise a portion of PE that spans residues 280-344 of domain II of PE. The amino acids at positions 339 and 343 appear to be necessary for transcytosis. See Siegall et al., 1991, Biochemistry 30:7154-59. Further, conservative or nonconservative substitutions can be made to the amino acid sequence of the transcytosis domain, as long as transcytosis activity is not substantially eliminated. A representative assay that can routinely be used by one of skill in the art to determine whether a transcytosis domain has transcytosis activity is described below.

Without intending to be limited to any particular theory or mechanism of action, the transcytosis domain is believed to permit the trafficking of the delivery construct through a polarized epithelial cell after the construct binds to a receptor present on the apical surface of the polarized epithelial cell. Such trafficking through a polarized epithelial cell is referred to herein as “transcytosis.” This trafficking permits the release of the delivery construct from the basal-lateral membrane of the polarized epithelial cell.

### 6.2.3. Particles for Delivery

The delivery constructs of the invention also comprise a particle to be delivered to a subject. The particle can be attached to the remainder of the delivery construct by any method known by one of skill in the art, without limitation. Further, the particle can be connected to any other portion of the delivery construct, without limitation, so long as the attachment does not disrupt the cell-binding activity and transcytosis activity of the other domains.

In certain embodiments, the particle can be connected with the N-terminal or C-terminal end of a polypeptide portion of the delivery construct. In such embodiments, the method of connection should be designed to avoid interference with other functions of the delivery construct, such as receptor binding or transcytosis. In yet other embodiments, the particle can be connected with a side chain of an amino acid of the delivery construct. The particle can be connected with the remainder of the delivery construct with a cleavable linker, as described below. In such embodiments, the particle to be delivered can be connected with the remainder of the delivery construct with one or more cleavable linkers such that cleavage at the cleavable linker(s) separates the particle from the remainder of the delivery construct. It should be noted that, in certain embodiments, the particle of interest can also comprise a short (1-50 amino acids, preferably 1-20 amino acids, more preferably 1-10 amino acids, and still more preferably 1-5 amino acids) leader peptide in addition to the particle of interest that remains attached to the particle following cleavage of the cleavable linker. Preferably, this leader peptide does not affect the activity or immunogenicity of the particle.

The particle can be any particle that is desired to be introduced into a subject. Thus, the particle can be a metal, a liposphere, a porous particle, a cell (either living or dead), a high-contrast particle, a coated particle, a peptide or polypeptide aggregate, a peptide or polypeptide crystal, or any combination thereof. In certain embodiments, the particle is a liposphere. In certain embodiments, the particle is a porous particle. In certain embodiments, the particle is a cell. In certain embodiments, the cell is a mammalian cell. In certain embodiments, the cell is a human, rat, mouse, dog, hamster, chicken, or monkey cell. In certain embodiments, the particle is a high-contrast particle. In certain embodiments, the particle is a peptide or polypeptide aggregate. In certain embodiments, the particle is a peptide or polypeptide crystal.

In certain embodiments, the particle comprises a metal. In certain embodiments, the particle is a metal particle. In certain embodiments, the particle is or comprises a metal selected from the group consisting Be, Mg, Ca, Sr, Ba, Ra, Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, Al, Ga, In, Sb, Pb, Te, Bi, larithanide metals, actinide metals, and alloys thereof. In certain embodiments, the particle is a platinum or gold particle. Guidance on making metal particles may be found, for example, in U.S. Pat. Nos. 6,755,886 and 6,689,192.

In certain embodiments, the particle can be a high-contrast particle. Thus, the particle can comprise a detectable compound such as a radiopaque compound, including air and barium and magnetic compounds. In certain embodiments, the particle can be either soluble or insoluble in water. In, for example, embodiments suitable for diagnostic applications, the particle can be conjugated to or can itself comprise a pharmaceutically acceptable gamma-emitting moiety, including but not limited to, indium and technetium, magnetic particles, radiopaque materials such as air or barium, and one or more fluorescent compound(s). Further guidance regarding construction and use of particles suitable for use in diagnostic or imaging applications, such as, e.g., high-contrast particles and detectably-labeled particles, can be found in U.S. Pat. Nos. 6,964,747, 6,919,068, 6,916,661, 6,800,765, 6,773,812, 6,540,981 and 6,159,445.

In certain embodiments, the particle can be a cell. In certain embodiments, the cell is a obtained from blood, pupils, irises, finger tips, teeth, portions of the skin, hair, mucous membranes, bladder, breast, male/female reproductive system components, muscle, vascular components, central nervous system components, liver, bone, colon, pancreas, or any other biological structure or organ known to one of skill in the art without limitation. In certain embodiments, the cell can be a human cell, non-human animal cell, plant cell, and synthetic/research cells. In certain embodiments, the cell can be a prokaryotic or a eukaryotic cell. In certain embodiments, the cell can be healthy, cancerous, mutated, damaged, diseased, or dead.

Any human cell known to one skilled in the art without limitation can be delivered with a delivery construct of the invention. Exemplary human cells include, but are not limited to, fibroblast cells, skeletal muscle cells, neutrophil white blood cells, lymphocyte white blood cells, erythroblast red blood cells, osteoblast bone cells, chondrocyte cartilage cells, basophil white blood cells, eosinophil white blood cells, adipocyte fat cells, neurons, adrenomedullary cells, melanocytes, epithelial cells, endothelial cells, cardiomyocytes, endothelial cells, epithelial cells, lymphocytes (T-cell and B cell), mast cells, eosinophils, vascular intimal cells, hepatocytes, leukocytes including mononuclear leukocytes, stem cells of any type, including haemopoetic, neural, skin, lung, kidney, liver and myocyte stem cells, osteoclasts, chondrocytes and other connective tissue cells, keratinocytes, melanocytes, liver cells, kidney cells, and adipocytes. Examples of research cells include transformed cells, Jurkat T cells, NIH3T3 cells, CHO, COS, etc. In certain embodiments, the cell can comprise genes not normally found in such cells, e.g., the cell can have one or more exogenous genes introduced into the cell prior to administration to the subject. Alternately, the cell can comprise one or more exogenous genetic elements that alters expression of genes found in the cell's genome. For example, the cell can comprise genetic elements that cause over-expression, regulable expression, under-expression, constitutive expression, etc. of a gene normally present in the cell's genome.

A useful source of cell lines and other biological material may be found in ATCC Cell Lines and Hybridomas, Bacteria and Bacteriophages, Yeast, Mycology and Botany, and Protists: Algae and Protozoa, and others available from American Type Culture Co. (Rockville, Md.), all of which are herein incorporated by reference.

In certain embodiments, the particle can be a particle that can perform a desirable biological activity when introduced to the bloodstream of the subject. For example, the particle can have receptor binding activity, enzymatic activity, messenger activity (i.e., act as a hormone, cytokine, neurotransmitter, or other signaling molecule), luminescent or other detectable activity, or regulatory activity, or any combination thereof. In other embodiments, the particle that is delivered can exert its effects in biological compartments of the subject other than the subject's blood. For example, in certain embodiments, the particle can exert its effects in the lymphatic system. In other embodiments, the particle can exert its effects in an organ or tissue, such as, for example, the subject's liver, heart, lungs, pancreas, kidney, brain, bone marrow, etc. In such embodiments, the particle may or may not be present in the blood, lymph, or other biological fluid at detectable concentrations, yet may still accumulate at sufficient concentrations at its site of action to exert a biological effect. In some embodiments, the particle can be an aggregate of a peptide or polypeptide having a desirable biological activity as described above. For example, the particle can be an aggregate of insulin, growth hormone, an interleukin, and the like. Exemplary peptides, proteins, cytokines, growth factors, hormones, enzymes, etc. are that can be used in such embodiments are extensively described below. In other embodiments, the particle can be a crystal comprising peptides or polypeptides assembled into a regular crystal lattice structure. For example, the crystal can be an insulin crystal comprising a plurality of insulin molecules assembled into a regular structure. Exemplary peptides, proteins, cytokines, growth factors, hormones, enzymes, etc. are that can be used in such embodiments are extensively described below.

In certain embodiments, the particle can be a liposphere or a porous particle. In certain embodiments, a liposphere can be a spherical aggregate with a diameter of about 0.1 to about 5 mm which contain at least one solid or liquid core surrounded by at least one continuous membrane. In another aspect, lipospheres can be finely dispersed liquid or solid phases coated with film-forming polymers, in the production of which the polymers are deposited onto the material to be encapsulated after emulsification and coacervation or interfacial polymerization. Porous particles can be made by absorbing liquid active principles in a matrix and may be optionally coated with film-forming polymers. The porous particles and lipospheres can be dried in the same way as powders. The particles can also contain two or more cores distributed in the continuous membrane material. In addition, single-core or multiple-core particles may be surrounded by an additional second, third etc. membrane.

The first, second, or additional membrane can each individually comprise natural, semisynthetic or synthetic materials. Natural membrane materials include, for example, gum arabic, agar agar, agarose, maltodextrins, alginic acid and salts thereof, for example, sodium or calcium alginate, fats and fatty acids, cetyl alcohol, collagen, chitosan, lecithins, gelatin, albumin, shellac, polysaccharides, such as starch or dextran, polypeptides, protein hydrolyzates, phospholipids, e.g., phosphatidylcholine, phosphatidylethanolamine, phosphatidylserine, phosphatidylinositol, phosphatidylglycerol, phosphatidic acid, lysophospholipids, egg or soybean phospholipid or any combination thereof, sucrose and waxes. Semisynthetic membrane materials include, e.g., chemically modified celluloses, e.g., cellulose esters and ethers, such as, for example, cellulose acetate, ethyl cellulose, hydroxypropyl cellulose, hydroxypropyl methyl cellulose and carboxymethyl cellulose, starch derivatives, e.g., starch ethers and esters, chitin derivatives, e.g., chitosan, and chemically modified phospholipids. Synthetic membrane materials include, for example, polymers, such as polyacrylates, polyamides, polyvinyl alcohol or polyvinyl pyrrolidone, and synthetic phospholipids.

The lipospheres and/or porous particles can also comprise a surfactant such as, e.g., natural surfactants such as casein, gelatin, tragacanth, waxes, enteric resins, paraffin, acacia, gelatin, cholesterol esters and triglycerides, (b) nonionic surfactants such as polyoxyethylene fatty alcohol ethers, sorbitan fatty acid esters, polyoxyethylene fatty acid esters, sorbitan esters, glycerol monostearate, polyethylene glycols, cetyl alcohol, cetostearyl alcohol, stearyl alcohol, poloxamers, polaxamines, methylcellulose, hydroxycellulose, hydroxy propylcellulose, hydroxy propylmethylcellulose, noncrystalline cellulose, polyvinyl alcohol, polyvinylpyrrolidone, and synthetic phospholipids, (c) anionic surfactants such as potassium laurate, triethanolamine stearate, sodium lauryl sulfate, alkyl polyoxyethylene sulfates, sodium alginate, dioctyl sodium sulfosuccinate, negatively charged phospholipids (phosphatidyl glycerol, phosphatidyl inosite, phosphatidylserine, phosphatidic acid and their salts), and negatively charged glyceryl esters, sodium carboxymethylcellulose, and calcium carboxymethylcellulose, (d) cationic surfactants such as quaternary ammonium compounds, benzalkonium chloride, cetyltrimethylammonium bromide, chitosans and lauryldimethylbenzylammonium chloride, (e) colloidal clays such as bentonite and veegum. Other suitable surfactants include, but are not limited to, one or a combination of the following: polaxomers, such as Pluronic™ F68, F108 and F127, which are block copolymers of ethylene oxide and propylene oxide available from BASF, and poloxamines, such as Tetronic™ 908 (T908), which is a tetrafunctional block copolymer derived from sequential addition of ethylene oxide and propylene oxide to ethylene-diamine available from BASF, Triton™ X-200, which is an alkyl aryl polyether sulfonate, available from Rohm and Haas, Tween 20, 40, 60 and 80, which are polyoxyethylene sorbitan fatty acid esters, available from ICI Specialty Chemicals, Carbowax™ 3550 and 934, which are polyethylene glycols available from Union Carbide, hydroxy propylmethylcellulose, dimyristoyl phosphatidylglycerol sodium salt, sodium dodecylsulfate, sodium deoxycholate, and cetyltrimethylammonium bromide.

Commercially available lipospheres and porous particles include Hallcrest Microcapsules (Hallcrest Inc., Glenview, Ill.); Thalaspheres (Engelhard Corp., Iselin, N.J.); Lipotec Millicapsules (Lipotec SA, Barcelona, Spain); Induchem Unispheres (Induchem SA, Volketswil, Switzerland); Glycospheres (Kobo Products Inc., South Plainfield, N.J.), and Softspheres (Kobo Products Inc., South Plainfield, N.J.).

Additional guidance regarding construction and use of lipospheres and/or porous particles for use in the delivery constructs of the invention may be found, for example, in U.S. Pat. Nos. 6,979,467; 6,974,593; 6,969,531; 6,969,530; 6,967,028; 6,953,593; 6,951,655; 6,949,239; 6,916,490; 6,884,432; 6,867,181; 6,862,890; 6,824,791; 6,794,364; 6,780,434; 6,790,460; 6,713,087; 6,685,960; 6,753,015; 6,749,866; 6,746,635; 6,682,761; 6,676,972; 6,416,740; 6,395,302; 6,245,349; 6,197,349; 5,885,486; 5,858,398; 5,672,358; 5,393,527; 5,246,707; 5,188,837; 5,091,188; 5,091,187; 4,725,442; and 4,622,219. In particular, U.S. Pat. No. 5,393,527 describes methods for coupling, e.g., the portion of the delivery construct comprising the receptor binding and translocation domains to the liposphere.

In certain embodiments, the particle can be a coated particle. For example, aqueous/solvent (wet/sol) techniques can be used to adhere polymeric coatings onto particulate materials. Suitable coatings include, but are not limited to, for example, biodegradable and biocompatible polymers, polysaccharides, and proteins. Suitable biodegradable polymers include, for example, poly(lactic acid) (PLA), poly(glycolic acid) (PGA), their copolymers poly(lactic-co-glycolic acid) (PLGA), and other polylactic acid polymers and copolymers, polyorthoesters, and polycaprolactones, etc. Suitable biocompatible polymers include, for example, polyethyleneglycols, polyvinylpyrrolidone, and polyvinylalcohols, etc. Suitable polysaccharides include, for example, dextrans, cellulose, xantham, chitins and chitosans, etc. Suitable proteins include, for example, polylysines and other polyamines, collagen, albumin, etc.

Further, the coated particles can comprise any solid substance known to one of skill in the art without limitation. For example, the particles can comprise, for example, one or more biologically active agent(s) to be administered to a subject, a metal particle, a glass particle, etc. Further guidance for construction and use of coated particles may be found, for example, in U.S. Pat. Nos. 6,984,404, 6,908,626, and 6,638,621, and in Zeng et al., 1995, Int. J. Pharm., 124:149-64.

In certain embodiments, the particles can comprise a biologically active agent for delivery to be administered to the subject. Such agents can be delivered with, for example, porous particles, coated particles, or lipospheres. Any biologically active agent known to one skilled in the art, without limitation, can be delivered with a particle. Examples of such biologically active agents that can be delivered with a particle according to the present invention include, but are not limited to, antineoplastic compounds, such as nitrosoureas, e.g., carmustine, lomustine, semustine, strepzotocin; methylhydrazines, e.g., procarbazine, dacarbazine; steroid hormones, e.g., glucocorticoids, estrogens, progestins, androgens, tetrahydrodesoxycaricosterone; immunoactive compounds such as immunosuppressives, e.g., pyrimethamine, trimethopterin, penicillamine, cyclosporine, azathioprine; and immunostimulants, e.g., levamisole, diethyl dithiocarbamate, enkephalins, endorphins; antimicrobial compounds such as antibiotics, e.g., β-lactam, penicillin, cephalosporins, carbapenims and monobactams, β-lactamase inhibitors, aminoglycosides, macrolides, tetracyclines, spectinomycin; antimalarials, amebicides; antiprotozoals; antifungals, e.g., amphotericin β, antivirals, e.g., acyclovir, idoxuridine, ribavirin, trifluridine, vidarabine, gancyclovir; parasiticides; antihalmintics; radiopharmaceutics; gastrointestinal drugs; hematologic compounds; immunoglobulins; blood clotting proteins, e.g., antihemophilic factor, factor IX complex; anticoagulants, e.g., dicumarol, heparin Na; fibrolysin inhibitors, e.g., tranexamic acid; cardiovascular drugs; peripheral anti-adrenergic drugs; centrally acting antihypertensive drugs, e.g., methyldopa, methyldopa HCl; antihypertensive direct vasodilators, e.g., diazoxide, hydralazine HCl; drugs affecting renin-angiotensin system; peripheral vasodilators, e.g., phentolamine; anti-anginal drugs; cardiac glycosides; inodilators, e.g., aminone, milrinone, enoximone, fenoximone, imazodan, sulmazole; antidysrhythmics; calcium entry blockers; drugs affecting blood lipids, e.g., ranitidine, bosentan, rezulin; respiratory drugs; sympathomimetic drugs, e.g., albuterol, bitolterol mesylate, dobutamine HCl, dopamine HCl, ephedrine So, epinephrine, fenfluramine HCl, isoproterenol HCl, methoxyamine HCl, norepinephrine bitartrate, phenylephrine HCl, ritodrine HCl; cholinomimetic drugs, e.g., acetylcholine Cl; anticholinesterases, e.g., edrophonium Cl; cholinesterase reactivators; adrenergic blocking drugs, e.g., acebutolol HCl, atenolol, esmolol HCl, labetalol HCl, metoprolol, nadolol, phentolamine mesylate, propranolol HCl; antimuscarinic drugs, e.g., anisotropine methylbromide, atropine SO₄, clinidium Br, glycopyrrolate, ipratropium Br, scopolamine HBr; neuromuscular blocking drugs; depolarizing drugs, e.g., atracurium besylate, hexafluorenium Br, metocurine iodide, succinylcholine Cl, tubocurarine Cl, vecuronium Br; centrally acting muscle relaxants, e.g., baclofen; neurotransmitters and neurotransmitter agents, e.g., acetylcholine, adenosine, adenosine triphosphate; amino acid neurotransmitters, e.g., excitatory amino acids, GABA, glycine; biogenic amine neurotransmitters, e.g., dopamine, epinephrine, histamine, norepinephrine, octopamine, serotonin, tyramine; neuropeptides, nitric oxide, K⁺ channel toxins; antiparkinson drugs, e.g., amaltidine HCl, benztropine mesylate, carbidopa; diuretic drugs, e.g., dichlorphenamide, methazolamide, bendroflumethiazide, polythiazide; antimigraine drugs, e.g, carboprost tromethamine mesylate, methysergide maleate.

Still other examples of biologically active agents that can be delivered with a particle according to the present invention include, but are not limited to, hormones such as pituitary hormones, e.g., chorionic gonadotropin, cosyntropin, menotropins, somatotropin, iorticotropin, protirelin, thyrotropin, vasopressin, lypressin; adrenal hormones, e.g., beclomethasone dipropionate, betamethasone, dexamethasone, triamcinolone; pancreatic hormones, e.g., glucagon, insulin; parathyroid hormone, e.g., dihydrochysterol; thyroid hormones, e.g., calcitonin etidronate disodium, levothyroxine Na, liothyronine Na, liotrix, thyroglobulin, teriparatide acetate; antithyroid drugs; estrogenic hormones; progestins and antagonists; hormonal contraceptives; testicular hormones; gastrointestinal hormones, e.g., cholecystokinin, enteroglycan, galanin, gastric inhibitory polypeptide, epidermal growth factor-urogastrone, gastric inhibitory polypeptide, gastrin-releasing peptide, gastrins, pentagastrin, tetragastrin, motilin, peptide YY, secretin, vasoactive intestinal peptide, sincalide.

Still other examples of biologically active agents that can be delivered with a particle according to the present invention include, but are not limited to, enzymes such as hyaluronidase, streptokinase, tissue plasminogen activator, urokinase, PGE-adenosine deaminase; intravenous anesthetics such as droperidol, etomidate, fetanyl citrate/droperidol, hexobarbital, ketamine HCl, methohexital Na, thiamylal Na, thiopental Na; antiepileptics, e.g., carbamazepine, clonazepam, divalproex Na, ethosuximide, mephenyloin, paramethadione, phenyloin, primidone.

Still other examples of biologically active agents that can be delivered with a particle according to the present invention include, but are not limited to, peptides and proteins such as heparin, ankyrins, arrestins, bacterial membrane proteins, clathrin, connexins, dystrophin, endothelin receptor, spectrin, selectin, cytokines; chemokines; growth factors, insulin, erythropoietin (EPO), tumor necrosis factor (TNF), neuropeptides, neuropeptide Y, neurotensin, transforming growth factor α, transforming growth factor β, interferon (IFN); hormones, growth inhibitors, e.g., genistein, steroids etc; glycoproteins, e.g., ABC transporters, platelet glycoproteins, GPIb-IX complex, GPIIb-IIIa complex, vitronectin, thrombomodulin, CD4, CD55, CD58, CD59, CD44, lymphocyte function-associated antigen, intercellular adhesion molecule, vascular cell adhesion molecule, Thy-1, antiporters, CA-15-3 antigen, fibronectins, laminin, myelin-associated glycoprotein, GAP, GAP-43.

Yet other examples of biologically active agents that can be delivered with a particle according to the present invention include, but are not limited to, cytokines and cytokine receptors such as Interleukin-1 (IL-1), IL-2, IL-3, IL-4, IL-5, IL-6, IL-7, IL-8, IL-9, IL-10, IL11, IL-12, IL-13, IL-14, IL-15, IL-16, IL-17, IL-18, IL-1 receptor, IL-2 receptor, IL-3 receptor, IL-4 receptor, IL-5 receptor, IL-6 receptor, IL-7 receptor, IL-8 receptor, IL-9 receptor, IL-10 receptor, IL-11 receptor, IL-12 receptor, IL-13 receptor, IL-14 receptor, IL-15 receptor, IL-16 receptor, IL-17 receptor, IL-18 receptor, lymphokine inhibitory factor, macrophage colony stimulating factor, platelet derived growth factor, stem cell factor, tumor growth factor β, tumor necrosis factor, lymphotoxin, Fas, granulocyte colony stimulating factor, granulocyte macrophage colony stimulating factor, interferon α, interferon β, and interferon γ.

Still other examples of biologically active agents that can be delivered with a particle according to the present invention include, but are not limited to, growth factors and protein hormones such as erythropoietin, angiogenin, hepatocyte growth factor, fibroblast growth factor, keratinocyte growth factor, nerve growth factor, tumor growth factor α, thrombopoietin, thyroid stimulating factor, thyroid releasing hormone, neurotrophin, epidermal growth factor, VEGF, ciliary neurotrophic factor, LDL, somatomedin, insulin growth factor, insulin-like growth factor I and II; chemokines such as ENA-78, ELC, GRO-α, GRO-β, GRO-γ, HRG, LIF, IP-10, MCP-1, MCP-2, MCP-3, MCP-4, MIP-1 α, MIP-1 β, MIG, MDC, NT-3, NT-4, SCF, LIF, leptin, RANTES, lymphotactin, eotaxin-1, eotaxin-2, TARC, TECK, WAP-1, WAP-2, GCP-1, GCP-2; α-chemokine receptors, e.g., CXCR1, CXCR2, CXCR3, CXCR4, CXCR5, CXCR6, CXCR7; and β-chemokine receptors, e.g., CCR1, CCR2, CCR3, CCR4, CCR5, CCR6, CCR7.

Yet other examples of biologically active agents that can be delivered with a particle according to the present invention include, but are not limited to, chemotherapeutics, such as chemotherapy or anti-tumor agents which are effective against various types of human cancers, including leukemia, lymphomas, carcinomas, sarcomas, myelomas etc., such as, for example, doxorubicin, mitomycin, cisplatin, daunorubicin, bleomycin, actinomycin D, and neocarzinostatin.

Still other examples of biologically active agents that can be delivered with a particle according to the present invention include, but are not limited to, antibodies such as anti-cluster of differentiation antigen CD-1 through CD-166 and the ligands or counter receptors for these molecules; anti-cytokine antibodies, e.g., anti-IL-1 through anti-IL-18 and the receptors for these molecules; anti-immune receptor antibodies; antibodies against T cell receptors, major histocompatibility complexes I and II, B cell receptors, selectin killer inhibitory receptors, killer activating receptors, OX-40, MadCAM-1, Gly-CAM1, integrins, cadherins, sialoadherens, Fas, CTLA-4, Fc γ-receptors, Fc α-receptors, Fc ε-receptors, Fc μ-receptors, and their ligands; anti-metalloproteinase antibodies, e.g., antibodies specific for collagenase, MMP-1 through MMP-8, TIMP-1, TIMP-2; anti-cell lysis/proinflammatory molecules, e.g., perforin, complement components, prostanoids, nitron oxide, thromboxanes; and anti-adhesion molecules, e.g., carcinoembryonic antigens, lamins, fibronectins.

Yet other examples of biologically active agents that can be delivered with a particle according to the present invention include, but are not limited to, antiviral agents such as reverse transcriptase inhibitors and nucleoside analogs, e.g., ddI, ddC, 3TC, ddA, AZT; protease inhibitors, e.g., Invirase, ABT-538; and inhibitors of RNA processing, e.g., ribavirin.

Further, specific examples of biologically active agents that can be delivered with the delivery constructs of the present invention include Capoten, Monopril, Pravachol, Avapro, Plavix, Cefzil, Duricef/Ultracef, Azactam, Videx, Zerit, Maxipime, VePesid, Paraplatin, Platinol, Taxol, UFT, Buspar, Serzone, Stadol NS, Estrace, Glucophage (Bristol-Myers Squibb); Ceclor, Lorabid, Dynabac, Prozac, Darvon, Permax, Zyprexa, Humalog, Axid, Gemzar, Evista (Eli Lily); Vasotec/Vaseretic, Mevacor, Zocor, Prinivil/Prinizide, Plendil, Cozaar/Hyzaar, Pepcid, Prilosec, Primaxin, Noroxin, Recombivax HB, Varivax, Timoptic/XE, Trusopt, Proscar, Fosamax, Sinemet, Crixivan, Propecia, Vioxx, Singulair, Maxalt, Ivermectin (Merck & Co.); Diflucan, Unasyn, Sulperazon, Zithromax, Trovan, Procardia XL, Cardura, Norvasc, Dofetilide, Feldene, Zoloft, Zeldox, Glucotrol XL, Zyrtec, Eletriptan, Viagra, Droloxifene, Aricept, Lipitor (Pfizer); Vantin, Rescriptor, Vistide, Genotropin, Micronase/Glyn./Glyb., Fragmin, Total Medrol, Xanax/alprazolam, Sermion, Halcion/triazolam, Freedox, Dostinex, Edronax, Mirapex, Pharmorubicin, Adriamycin, Camptosar, Remisar, Depo-Provera, Caverject, Detrusitol, Estring, Healon, Xalatan, Rogaine (Pharmacia & Upjohn); Lopid, Accrupil, Dilantin, Cognex, Neurontin, Loestrin, Dilzem, Fempatch, Estrostep, Rezulin, Lipitor, Omnicef, FemHRT, Suramin, and Clinafloxacin (Warner Lambert).

Still further examples of biologically active agents which may be delivered with a particle by the delivery constructs of the present invention include nucleic acids encoding gene products for use in genetic therapies. Exemplary description of such particles can be found in U.S. Pat. Nos. 6,743,444; 6,696,423; 6,677,313; and 6,667,294.

In certain embodiments, the particle does not comprise a polypeptide. In certain embodiments, the particle is not a polypeptide. In certain embodiments, the particle does not comprise a complex of polypeptides. In certain embodiments, the particle is not a complex of polypeptides.

Yet further examples of biologically active agents which may be delivered with a particle by the delivery constructs of the present invention may be found in: Goodman and Gilman's The Pharmacological Basis of Therapeutics, 11th ed. McGraw-Hill 2005, incorporated herein by reference in its entirety.

In the discussion that follows, the sizes of particles suitable for use in the delivery constructs of the invention are described. The description of the sizes in terms of the diameter of the particle by no means mandates that the particles be either roughly or perfectly spherical. Indeed, it is contemplated that the particles can be of any shape without limitation. The sizes of the particles are described in terms of diameter merely for convenience. In the cases of particles of other shape, the longest dimension of the particle should be used in place of the distance labeled as diameter in the discussion that follows. Thus, for roughly cubical particles, the longest edge of the particle should be considered a “diameter.”

In certain embodiments, the particle is between about 0.1 nm and about 150 nm in diameter. In certain embodiments, the particle is between about 1 nm and about 150 nm in diameter. In certain embodiments, the particle is between about 10 nm and about 150 nm in diameter. In certain embodiments, the particle is between about 25 nm and about 150 nm in diameter. In certain embodiments, the particle is between about 50 nm and about 150 nm in diameter. In certain embodiments, the particle is between about 75 nm and about 150 nm in diameter. In certain embodiments, the particle is between about 100 nm and about 150 nm in diameter. In certain embodiments, the particle is between about 125 nm and about 150 nm in diameter.

In certain embodiments, the particle is between about 0.1 nm and about 125 nm in diameter. In certain embodiments, the particle is between about 1 nm and about 125 nm in diameter. In certain embodiments, the particle is between about 10 nm and about 125 nm in diameter. In certain embodiments, the particle is between about 25 nm and about 125 nm in diameter. In certain embodiments, the particle is between about 50 nm and about 125 nm in diameter. In certain embodiments, the particle is between about 75 nm and about 125 nm in diameter. In certain embodiments, the particle is between about 100 nm and about 125 nm in diameter.

In certain embodiments, the particle is between about 0.1 nm and about 100 nm in diameter. In certain embodiments, the particle is between about 1 nm and about 100 nm in diameter. In certain embodiments, the particle is between about 10 nm and about 100 nm in diameter. In certain embodiments, the particle is between about 25 nm and about 100 nm in diameter. In certain embodiments, the particle is between about 50 nm and about 100 nm in diameter. In certain embodiments, the particle is between about 75 nm and about 100 nm in diameter.

In certain embodiments, the particle is between about 0.1 nm and about 75 nm in diameter. In certain embodiments, the particle is between about 1 nm and about 75 nm in diameter. In certain embodiments, the particle is between about 10 nm and about 75 nm in diameter. In certain embodiments, the particle is between about 25 nm and about 75 nm in diameter. In certain embodiments, the particle is between about 50 nm and about 75 nm in diameter.

In certain embodiments, the particle is between about 0.1 nm and about 50 nm in diameter. In certain embodiments, the particle is between about 1 nm and about 50 nm in diameter. In certain embodiments, the particle is between about 10 nm and about 50 nm in diameter. In certain embodiments, the particle is between about 25 nm and about 50 nm in diameter. In certain embodiments, the particle is between about 0.1 nm and about 25 nm in diameter. In certain embodiments, the particle is between about 1 nm and about 25 nm in diameter. In certain embodiments, the particle is between about 10 nm and about 25 nm in diameter. In certain embodiments, the particle is between about 0.1 nm and about 10 nm in diameter. In certain embodiments, the particle is between about 1 nm and about 10 nm in diameter. In certain embodiments, the particle is between about 0.1 nm and about 1 nm in diameter.

In certain embodiments, the particle is smaller than a polarized epithelial cell. In certain embodiments, the particle is about 10% smaller than a polarized epithelial cell. In certain embodiments, the particle is about 15% smaller than a polarized epithelial cell. In certain embodiments, the particle is about 20% smaller than a polarized epithelial cell. In certain embodiments, the particle is about 25% smaller than a polarized epithelial cell. In certain embodiments, the particle is about 30% smaller than a polarized epithelial cell. In certain embodiments, the particle is about 35% smaller than a polarized epithelial cell. In certain embodiments, the particle is about 40% smaller than a polarized epithelial cell. In certain embodiments, the particle is about 45% smaller than a polarized epithelial cell. In certain embodiments, the particle is about 50% smaller than a polarized epithelial cell. In certain embodiments, the particle is about 55% smaller than a polarized epithelial cell. In certain embodiments, the particle is about 60% smaller than a polarized epithelial cell. In certain embodiments, the particle is about 65% smaller than a polarized epithelial cell. In certain embodiments, the particle is about 70% smaller than a polarized epithelial cell. In certain embodiments, the particle is about 75% smaller than a polarized epithelial cell. In certain embodiments, the particle is about 80% smaller than a polarized epithelial cell. In certain embodiments, the particle is about 85% smaller than a polarized epithelial cell. In certain embodiments, the particle is about 90% smaller than a polarized epithelial cell. In certain embodiments, the particle is about 95% smaller than a polarized epithelial cell. In certain embodiments, the particle is about 98% smaller than a polarized epithelial cell. In certain embodiments, the particle is about 99% smaller than a polarized epithelial cell. In certain embodiments, the polarized epithelial cell is a mammalian epithelial cell. In certain embodiments, the polarized epithelial cell is a primate epithelial cell. In certain embodiments, the polarized epithelial cell is a human, mouse, or rat epithelial cell. In certain embodiments, the polarized epithelial cell is a human epithelial cell.

In certain embodiments, the particle can be inactive or in a less active form when administered, then be activated in the subject. For example, the particle can comprise a peptide or polypeptide with a masked active site. The peptide or polypeptide can be activated by removing the masking moiety. Such removal can be accomplished by peptidases or proteases in the cases of peptide or polypeptide masking agents. Alternatively, the masking agent can be a chemical moiety that is removed by an enzyme present in the subject. This strategy can be used when it is desirable for the particle to be active in limited circumstances. For example, it may be useful for a particle have an activity, such as, for example, receptor binding, only in the liver of the subject. In such cases, the particle can comprise a binding agent that has a masking moiety that can be removed by an enzyme that is present in the liver, but not in other organs or tissues. Exemplary methods and compositions for making and using such masked binding agents for use in particles can be found in U.S. Pat. Nos. 6,080,575, 6,265,540, and 6,670,147.

As discussed above, any suitable method known to one skilled in the art, without limitation, can be used to couple the particle to be delivered to the remainder of the delivery construct. Generally, the method chosen will depend on the nature of the particle to be delivered. The method chosen will preferably connect the particle with the remainder of the delivery construct in a manner that does not prevent the receptor binding domain and the transcytosis domain from performing their functions. Further, in embodiments where a cleavable linker is used to connect the particle with the remainder of the delivery construct, the method used to couple the particle preferably specifically connects the particle with the remainder of the delivery construct at a position distal to the cleavable linker relative to the receptor binding and/or transcytosis domains.

Many suitable methods useful for coupling the particle to the remainder of the delivery construct are known to those skilled in the art. For example, the particle can be coupled with the remainder of the delivery construct through ionic or hydrophobic interactions between the two elements. In such embodiments, a sufficient amount of the polypeptide portion of the delivery construct can be absorbed onto the particle to ensure that the particle has active and available receptor binding domain(s) and transcytosis domain(s) attached thereto to permit transcytosis. Methods for testing the function of such domains is extensively described below. In addition, non-covalent coupling of particles to the remainder of the delivery construct can also be achieved by coating the particle with (or integrating into the particles with an exposure at the particle surface) materials such as proteins, peptides and sugars that would participate in non-covalent, non-specific interactions. These modifications can be made not only to the particles but also to the remainder of the delivery construct. Alternately, proteins, peptides, and/or sugars can be used that specifically bind a cognate binding partner. For example, the particle can be chemically coupled to biotin, the remainder of the delivery construct can have streptavidin attached to it, and the biotin-streptavidin linkage can connect the particle with the remainder of the delivery construct.

Further, chemical coupling of particles and carrier constructs can be performed randomly by linker chemistries such as, for example, those that reduce Schiff base structures formed by primary amine and carboxylic acid moieties in reductive deamidation reactions. Other chemistries can also be used if the molecules in the particle or the remainder of the delivery construct, particularly those that comprise sugar moieties. For example, the methods described in U.S. Pat. No. 5,889,155, specifically incorporated herein by reference in its entirety, can be used. In these methods, a nucleophilic hydrazide residue is reacted with an electrophilic maleimide residue, allowing coupling in one example, of aldehydes to free thiols. The cross-linking reagent can be modified to cross-link various functional groups and is thus useful for cross-linking polypeptides and sugars.

Additional exemplary methods suitable for connecting the particles to the remainder of the delivery construct are described in U.S. Pat. No. 5,603,872 and U.S. Pat. No. 5,401,511, each specifically incorporated herein by reference in its entirety. Thus, various ligands can be covalently bound to particle surfaces through the cross-linking of amine residues. In embodiments where the particle is a liposphere, for example, multilamellar vesicles, unilamellar vesicles such as microemulsified lipospheres, and large unilamellar lipospheres, each containing phosphatidylethanolamine, can be prepared by established procedures. The inclusion of phosphatidylethanolamine in the liposphere provides an active functional residue, a primary amine, on the liposphere surface for cross-linking purposes. Ligands can be bound covalently to discrete sites on the liposphere surfaces. The number and surface density of these sites will be dictated by the liposphere formulation and the liposphere type. The liposphere surfaces may also have sites for non-covalent association. To form covalent conjugates of ligands and liposphere, cross-linking reagents, such as, for example, glutaraldehyde, bifunctional oxirane, ethylene glycol diglycidyl ether, and a water soluble carbodiimide, such as 1-ethyl-3-(3-dimethylaminopropyl)carbodiimide. Through cross-linking, the amine residues of the polypeptide portion of the delivery construct can be linked to the particle. These methods can also routinely be adapted for use with particles that are not lipospheres that also comprise a free primary amine on the particle surface.

### 6.2.4. Cleavable Linkers

In certain embodiments of the delivery constructs of the invention, the particle to be delivered to the subject can be optionally connected with the remainder of the delivery construct with one or more cleavable linkers. When the particle can be separated from the remainder of the delivery construct with cleavage at a single linker, the delivery constructs can comprise a single cleavable linker. Alternately, the particle can be connected with the remainder of the delivery construct with two or more cleavable linkers.

In certain embodiments, the cleavable linkers can be cleavable by a cleaving enzyme that is present at or near the basal-lateral membrane of an epithelial cell. By selecting the cleavable linker to be cleaved by such enzymes, the particle can be liberated from the remainder of the construct following transcytosis across the mucous membrane and release from the epithelial cell into the cellular matrix on the basal-lateral side of the membrane. Further, cleaving enzymes can be used that are present inside the epithelial cell, such that the cleavable linker is cleaved prior to release of the delivery construct from the basal-lateral membrane, so long as the cleaving enzyme does not cleave the delivery construct before the delivery construct enters the trafficking pathway in the polarized epithelial cell that results in release of the delivery construct and particle from the basal-lateral membrane of the cell.

In certain embodiments, the cleaving enzyme is a peptidase. In other embodiments, the cleaving enzyme is an RNAse. In yet other embodiments, the cleaving enzyme can cleave carbohydrates. Preferred peptidases include, but are not limited to, Cathepsin GI, Chymotrypsin I, Elastase I, Subtilisin AI, Subtilisin AII, Thrombin I, and Urokinase I. Table 1 presents these enzymes together with an amino acid sequence that is recognized and cleaved by the particular peptidase.

TABLE 1

Peptidases Present Near Basal-Lateral Mucous
Membranes

| | |
|---|---|
| | Amino Acid Sequence Recognized and |
| Peptidase | Cleaved |

| | | |
|---|---|---|
| Cathepsin GI | Ala-Ala-Pro-Phe | (SEQ ID NO.: 4) |
| Chymotrypsin I | Gly-Gly-Phe | (SEQ ID NO.: 5) |
| Elastase I | Ala-Ala-Pro-Val | (SEQ ID NO.: 6) |
| Subtilisin AI | Gly-Gly-Leu | (SEQ ID NO.: 7) |
| Subtilisin AII | Ala-Ala-Leu | (SEQ ID NO.: 8) |
| Thrombin I | Phe-Val-Arg | (SEQ ID NO.: 9) |
| Urokinase I | Val-Gly-Arg | (SEQ ID NO.: 10) |

In certain embodiments, the delivery construct can comprise more than one cleavable linker, wherein cleavage at either cleavable linker can separate the particle to be delivered from the delivery construct. In certain embodiments, the cleavable linker can be selected based on the sequence, in the case of particles that comprise peptides, polypeptides, or proteins, to avoid the use of cleavable linkers that comprise sequences present in the particle to be delivered. For example, if the particle comprises AAL, the cleavable linker can be selected to be cleaved by an enzyme that does not recognize this sequence.

Further, the cleavable linker preferably exhibits a greater propensity for cleavage than the remainder of the delivery construct. As one skilled in the art is aware, many peptide and polypeptide sequences can be cleaved by peptidases and proteases. In certain embodiments, the cleavable linker is selected to be preferentially cleaved relative to other amino acid sequences present in the delivery construct during administration of the delivery construct. In certain embodiments, the receptor binding domain is substantially (e.g., about 99%, about 95%, about 90%, about 85%, about 80, or about 75%) intact following delivery of the delivery construct to the bloodstream of the subject. In certain embodiments, the translocation domain is substantially (e.g., about 99%, about 95%, about 90%, about 85%, about 80, or about 75%) intact following delivery of the delivery construct to the bloodstream of the subject. In certain embodiments, the particle is substantially (e.g., about 99%, about 95%, about 90%, about 85%, about 80, or about 75%) intact following delivery of the delivery construct to the bloodstream of the subject. In certain embodiments, the cleavable linker is substantially (e.g., about 99%, about 95%, about 90%, about 85%, about 80, or about 75%) cleaved following delivery of the delivery construct to the bloodstream of the subject.

In other embodiments, the cleavable linker is cleaved by a cleaving enzyme found in the plasma of the subject. Any cleaving enzyme known by one of skill in the art to be present in the plasma of the subject can be used to cleave the cleavable linker. Use of such enzymes to cleave the cleavable linkers is less preferred than use of cleaving enzymes found near the basal-lateral membrane of a polarized epithelial cell because it is believed that more efficient cleavage will occur in near the basal-lateral membrane. However, if the skilled artisan determines that cleavage mediated by a plasma enzyme is sufficiently efficient to allow cleavage of a sufficient fraction of the delivery constructs to avoid adverse effects, such plasma cleaving enzymes can be used to cleave the delivery constructs. Accordingly, in certain embodiments, the cleavable linker can be cleaved with an enzyme that is selected from the group consisting of caspase-1, caspase-3, proprotein convertase 1, proprotein convertase 2, proprotein convertase 4, proprotein convertase 4 PACE 4, prolyl oligopeptidase, endothelin cleaving enzyme, dipeptidyl-peptidase IV, signal peptidase, neprilysin, renin, and esterase. See, e.g., U.S. Pat. No. 6,673,574. Table 2 presents these enzymes together with an amino acid sequence(s) recognized by the particular peptidase. The peptidase cleaves a peptide comprising these sequences at the N-terminal side of the amino acid identified with an asterisk.

TABLE 2

Plasma Peptidases

| | Amino Acid Sequence Recog- |
|---|---|
| Peptidase | nized and Cleaved |
| Caspase-1 | Tyr-Val-Ala-Asp-Xaa* |
| | (SEQ ID NO.: 11) |
| Caspase-3 | Asp-Xaa-Xaa-Asp-Xaa* |
| | (SEQ ID NO.: 12) |
| Proprotein convertase | Arg-(Xaa)ₙ-Arg-Xaa*; |
| 1 | n = 0, 2, 4 or 6 |
| | (SEQ ID NO.: 13) |
| Proprotein convertase | Lys-(Xaa)ₙ-Arg-Xaa*; |
| 2 | n = 0, 2, 4, or 6 |
| | (SEQ ID NO.: 14) |
| Proprotein convertase | Glp-Arg-Thr-Lys-Arg-Xaa* |
| 4 | (SEQ ID NO.: 15) |
| Proprotein convertase | Arg-Val-Arg-Arg-Xaa* |
| 4 PACE 4 | (SEQ ID NO.: 16) |
| | Decanoyl-Arg-Val-Arg-Arg-Xaa* |
| | (SEQ ID NO.: 17) |
| Prolyloligopeptidase | Pro-Xaa*-Trp-Val-Pro-Xaa |
| Endothelin cleaving | (SEQ ID NO.: 18) |
| enzyme in combination | |
| with dipeptidyl- | |
| peptidase IV | |
| Signal peptidase | Trp-Val*-Ala-Xaa |
| | (SEQ ID NO.: 19) |
| Neprilysin in combin- | Xaa-Phe*-Xaa˜Xaa |
| ation with dipep- | (SEQ ID NO.: 20) |
| tidyl-peptidase IV | Xaa-Tyr*-Xaa-Xaa |
| | (SEQ ID NO.: 21) |
| | Xaa-Trp*-Xaa˜Xaa |
| | (SEQ ID NO.: 22) |
| Renin in combination | Asp-Arg-Tyr-Ile-Pro-Phe-His- |
| with dipeptidyl- | Leu*-Leu-(Val, Ala or Pro)- |
| peptidase IV | Tyr-(Ser, Pro, or Ala) |
| | (SEQ ID NO.: 23) |

Thus, in certain more preferred embodiments, the cleavable linker can be any cleavable linker known by one of skill in the art to be cleavable by an enzyme that is present at the basal-lateral membrane of an epithelial cell. In certain embodiments, the cleavable linker comprises a peptide. In other embodiments, the cleavable linker comprises a nucleic acid, such as RNA or DNA. In still other embodiments, the cleavable linker comprises a carbohydrate, such as a disaccharide or a trisaccharide. In certain embodiments, the cleavable linker is a peptide that comprises an amino acid sequence that is selected from the group consisting of Ala-Ala-Pro-Phe (SEQ ID NO.:4), Gly-Gly-Phe (SEQ ID NO.:5), Ala-Ala-Pro-Val (SEQ ID NO.:6), Gly-Gly-Leu (SEQ ID NO.:7), Ala-Ala-Leu (SEQ ID NO.:8), Phe-Val-Arg (SEQ ID NO.:9), Val-Gly-Arg (SEQ ID NO.: 10).

Alternatively, in less preferred embodiments, the cleavable linker can be any cleavable linker known by one of skill in the art to be cleavable by an enzyme that is present in the plasma of the subject to whom the delivery construct is administered. In certain embodiments, the cleavable linker comprises a peptide. In other embodiments, the cleavable linker comprises a nucleic acid, such as RNA or DNA. In still other embodiments, the cleavable linker comprises a carbohydrate, such as a disaccharide or a trisaccharide. In certain embodiments, the cleavable linker is a peptide that comprises an amino acid sequence that is selected from the group consisting of amino acid sequences presented in Table 2.

In certain embodiments, the delivery construct comprises more than one cleavable linker. In certain embodiments, cleavage at any of the cleavable linkers will separate the particle to be delivered from the remainder of the delivery construct. In certain embodiments, the delivery construct comprises a cleavable linker cleavable by an enzyme present at the basal-lateral side of a polarized epithelial membrane and a cleavable linkers cleavable by an enzyme that is present in the plasma of the subject to whom the delivery construct is administered.

Further guidance regarding cleavable linkers that can be used in delivery constructs of the present invention, as well as assays for identifying and testing such linkers, can be found in U.S. application Ser. No. 11/244,349, filed Oct. 4, 2005, which is hereby incorporated by reference in its entirety.

### 6.3. Methods for Delivering a Particle

In another aspect, the invention provides methods for local or systemic delivery of a particle to a subject. These methods generally comprise administering a delivery construct of the invention to a mucous membrane of the subject to whom the particle is delivered. The delivery construct is typically administered in the form of a pharmaceutical composition, as described below.

Thus, in certain aspects, the invention provides a method for delivering a particle to a subject. The method comprises contacting an apical surface of a polarized epithelial cell of the subject with a delivery construct. In certain embodiments, the delivery construct comprises a receptor binding domain, a transcytosis domain, a particle to be delivered, and, optionally, a cleavable linker. The transcytosis domain can transcytose the particle to and through the basal-lateral membrane of the epithelial cell.

In certain embodiments, the receptor binding domain is selected from the group consisting of receptor binding domains from Pseudomonas exotoxin A, cholera toxin, diphtheria toxin, shiga toxin, or shiga-like toxin; monoclonal antibodies; polyclonal antibodies; single-chain antibodies; TGF α; EGF; IGF-I; IGF-II; IGF-III; IL-1; IL-2; IL-3; IL-6; MIP-1a; MIP-1b; MCAF; and IL-8. In certain embodiments, the receptor binding domain binds to a cell surface receptor selected from the group consisting of α2-macroglobulin receptor, EGFR, IGFR, transferrin receptor, chemokine receptor, CD25, CD11B, CD11C, CD80, CD86, TNFα receptor, TOLL receptor, M-CSF receptor, GM-CSF receptor, scavenger receptor, and VEGF receptor.

In certain embodiments, the transcytosis domain is selected from the group consisting of transcytosis domains from Pseudomonas exotoxin A, diphtheria toxin, pertussis toxin, cholera toxin, heat-labile E. coli enterotoxin, shiga toxin, and shiga-like toxin.

In certain embodiments, the particle can be a metal, a liposphere, a porous particle, a cell (either living or dead), a high-contrast particle, a peptide or polypeptide aggregate, a peptide or polypeptide crystal, or any combination thereof. In certain embodiments, the particle is a platinum or gold particle. In certain embodiments, the particle is a liposphere. In certain embodiments, the particle is a porous particle. In certain embodiments, the particle is a cell. In certain embodiments, the cell is a mammalian cell. In certain embodiments, the cell is a human, rat, mouse, dog, hamster, chicken, or monkey cell. In certain embodiments, the particle is a high-contrast particle. In certain embodiments, the particle is a peptide or polypeptide aggregate. In certain embodiments, the particle is a peptide or polypeptide crystal.

The optional cleavable linker can be cleaved by an enzyme that is present at a basal-lateral membrane of a polarized epithelial cell of the subject or in the plasma of the subject. Cleavage at the optional cleavable linker separates the particle from the remainder of the delivery construct, thereby delivering the particle to the subject.

In certain embodiments, the enzyme that is present at or near a basal-lateral membrane of a polarized epithelial cell is selected from the group consisting of Cathepsin GI, Chymotrypsin I, Elastase I, Subtilisin AI, Subtilisin AII, Thrombin I, and Urokinase I. In certain embodiments, the cleavable linker comprises an amino acid sequence that is selected from the group consisting of Ala-Ala-Pro-Phe (SEQ ID NO.:4), Gly-Gly-Phe (SEQ ID NO.:5), Ala-Ala-Pro-Val (SEQ ID NO.:6), Gly-Gly-Leu (SEQ ID NO.:7), Ala-Ala-Leu (SEQ ID NO.:8), Phe-Val-Arg (SEQ ID NO.:9), Val-Gly-Arg (SEQ ID NO.: 10).

In certain embodiments, the epithelial cell is selected from the group consisting of nasal epithelial cells, oral epithelial cells, intestinal epithelial cells, rectal epithelial cells, vaginal epithelial cells, and pulmonary epithelial cells.

In certain embodiments, the subject is a mammal. In further embodiments, the subject is a rodent, a lagomorph, or a primate. In yet further embodiments, the rodent is a mouse or rat. In other embodiments, the lagomorph is a rabbit. In still other embodiments, the primate is a human, monkey, or ape. In a preferred embodiment, the subject is a human.

In certain embodiments, the invention provides a method for delivering a particle to the bloodstream of a subject that results in at least about 30% bioavailability of the particles, comprising administering a delivery construct comprising the particles to the subject, thereby delivering at least about 30% of the total particles administered to the blood of the subject in a bioavailable form of the particle. In certain embodiments, at least about 10% of the total particles administered are bioavailable to the subject. In certain embodiments, at least about 15% of the total particles administered are bioavailable to the subject. In certain embodiments, at least about 20% of the total particles administered are bioavailable to the subject. In certain embodiments, at least about 25% of the total particles administered are bioavailable to the subject. In certain embodiments, at least about 35% of the total particles administered are bioavailable to the subject. In certain embodiments, at least about 40% of the total particles administered are bioavailable to the subject. In certain embodiments, at least about 45% of the total particles administered are bioavailable to the subject. In certain embodiments, at least about 50% of the total particles administered are bioavailable to the subject. In certain embodiments, at least about 55% of the total particles administered are bioavailable to the subject. In certain embodiments, at least about 60% of the total particles administered are bioavailable to the subject. In certain embodiments, at least about 65% of the total particles administered are bioavailable to the subject. In certain embodiments, at least about 70% of the total particles administered are bioavailable to the subject. In certain embodiments, at least about 75% of the total particles administered are bioavailable to the subject. In certain embodiments, at least about 80% of the total particles administered are bioavailable to the subject. In certain embodiments, at least about 85% of the total particles administered are bioavailable to the subject. In certain embodiments, at least about 90% of the total particles administered are bioavailable to the subject. In certain embodiments, at least about 95% of the total particles administered are bioavailable to the subject. In certain embodiments, the percentage of bioavailability of the particles is determined by comparing the amount of particles present in a subject's blood following administration of a delivery construct comprising the particles to the amount of particles present in a subject's blood following administration of the particles through another route of administration. In certain embodiments, the other route of administration is injection, e.g., subcutaneous injection, intravenous injection, intra-arterial injection, etc. In other embodiments, the percentage of bioavailability of the particles is determined by comparing the amount of particles present in a subject's blood following administration of a delivery construct comprising the particles to the total amount of particles administered as part of the delivery construct. In still other embodiments, the percentage of bioavailability of the particles is determined by comparing the amount of biologically active agent that is present in a subject's blood following administration of a delivery construct comprising a particle that comprises the biologically active agent(s) to the amount of biologically active agent present in a subject's blood following administration of the biologically active agent through another route of administration. In yet other embodiments, the percentage of bioavailability of the particles is determined by comparing the amount of biologically active agent that is present in a subject's blood following administration of a delivery construct comprising a particle that comprises the biologically active agent(s) to the total amount of biologically active agent administered as part of the delivery construct.

In certain embodiments, peak plasma concentrations of the delivered particle in the subject are achieved about 10 minutes after administration. In certain embodiments, peak plasma concentrations of the delivered particle in the subject are achieved about 15 minutes after administration. In certain embodiments, peak plasma concentrations of the delivered particle in the subject are achieved about 5 minutes after administration. In certain embodiments, peak plasma concentrations of the delivered particle in the subject are achieved about 20 minutes after administration. In certain embodiments, peak plasma concentrations of the delivered particle in the subject are achieved about 25 minutes after administration. In certain embodiments, peak plasma concentrations of the delivered particle in the subject are achieved about 30 minutes after administration. In certain embodiments, peak plasma concentrations of the delivered particle in the subject are achieved about 35 minutes after administration. In certain embodiments, peak plasma concentrations of the delivered particle in the subject are achieved about 40 minutes after administration. In certain embodiments, peak plasma concentrations of the delivered particle in the subject are achieved about 45 minutes after administration. In certain embodiments, peak plasma concentrations of the delivered particle in the subject are achieved about 50 minutes after administration. In certain embodiments, peak plasma concentrations of the delivered particle in the subject are achieved about 55 minutes after administration. In certain embodiments, peak plasma concentrations of the delivered particle in the subject are achieved about 60 minutes after administration. In certain embodiments, peak plasma concentrations of the delivered particle in the subject are achieved about 90 minutes after administration. In certain embodiments, peak plasma concentrations of the delivered particle in the subject are achieved about 120 minutes after administration. In embodiments where the particles comprise one or more biologically active agent(s), the peak plasma concentration of the particle can be measured by determining the concentration of the particles or the one or more biologically active agent(s) delivered as part of the delivery construct.

In certain embodiments, the peak plasma concentration of the delivered particle is between about 0.01 ng/ml plasma and about 10 μg/ml plasma. In certain embodiments, the peak plasma concentration of the delivered particle is between about 0.01 ng/ml plasma and about 1 μg/ml plasma. In certain embodiments, the peak plasma concentration of the delivered particle is between about 0.01 ng/ml plasma and about 0.1 μg/ml plasma. In certain embodiments, the peak plasma concentration of the delivered particle is between about 0.01 ng/ml plasma and about 10 ng/ml plasma. In certain embodiments, the peak plasma concentration of the delivered particle is between about 1 ng/ml plasma and about 10 μg/ml plasma. In certain embodiments, the peak plasma concentration of the delivered particle is between about 1 ng/ml plasma and about 0.1 μg/ml plasma. In certain embodiments, the peak plasma concentration of the delivered particle is between about 1 ng/ml plasma and about 0.5 μg/ml plasma. In certain embodiments, the peak plasma concentration of the delivered particle is between about 1 ng/ml plasma and about 0.1 μg/ml plasma. In certain embodiments, the peak plasma concentration of the delivered particle is between about 10 ng/ml plasma and about 1 μg/ml plasma. In certain embodiments, the peak plasma concentration of the delivered particle is between about 10 ng/ml plasma and about 0.5 μg/ml plasma.

In certain embodiments, the peak plasma concentration of the delivered particle is at least about 10 μg/ml plasma. In certain embodiments, the peak plasma concentration of the delivered particle is at least about 5 μg/ml plasma. In certain embodiments, the peak plasma concentration of the delivered particle is at least about 1 μg/ml plasma. In certain embodiments, the peak plasma concentration of the delivered particle is at least about 500 ng/ml plasma. In certain embodiments, the peak plasma concentration of the delivered particle is at least about 250 ng/ml plasma. In certain embodiments, the peak plasma concentration of the delivered particle is at least about 100 ng/ml plasma. In certain embodiments, the peak plasma concentration of the delivered particle is at least about 50 ng/ml plasma. In certain embodiments, the peak plasma concentration of the delivered particle is at least about 10 ng/ml plasma. In certain embodiments, the peak plasma concentration of the delivered particle is at least about 5 ng/ml plasma. In certain embodiments, the peak plasma concentration of the delivered particle is at least about 1 ng/ml plasma. In certain embodiments, the peak plasma concentration of the delivered particle is at least about 0.1 ng/ml plasma.

Moreover, without intending to be bound to any particular theory or mechanism of action, it is believed that oral administration of a delivery construct can deliver a higher effective concentration of the delivered particle (or of a biologically active agent delivered as part of the particle) to the liver of the subject than is observed in the subject's plasma. “Effective concentration,” in this context, refers to the concentration of either particles or biologically active agents delivered as part of a particle experienced by targets of the particles or biologically active agents and can be determined by monitoring and/or quantifying downstream effects of particle-target interactions. While still not bound to any particular theory, it is believed that oral administration of the delivery construct results in absorption of the delivery construct through polarized epithelial cells of the digestive mucosa, e.g., the intestinal mucosa, followed by cleavage of the construct and release of the particle at the basolateral side of the mucous membrane. As one of skill in the art will recognize, the blood at the basolateral membrane of such digestive mucosa is carried from this location to the liver via the portal venous system. Thus, when the particle exerts a biological activity in the liver, such as, for example, activities mediated by growth hormone, insulin, IGF-I, etc. binding to their cognate receptors, the particle is believed to exert an effect in excess of what would be expected based on the plasma concentrations observed in the subject. Accordingly, in certain embodiments, the invention provides a method of administering a particle to a subject that comprises orally administering a delivery construct comprising the particle to the subject, wherein the particle is delivered to the subject's liver at a higher effective concentration than observed in the subject's plasma.

In another aspect, the invention provides a method for delivering a particle to the bloodstream of a subject that induces a lower titer of antibodies against the particle than other routes of administration. Without intending to be bound by any particular theory or mechanism of action, it is believed that entry of the particle through a mucous membrane, e.g., through the intestinal mucosa, causes the immune system to tolerate the particle better than if the particle were, for example, injected. Thus, a lower titer of antibodies against the particle can be produced in the subject by delivering the particle with a delivery construct of the invention through the mucosa rather than injecting the particle, for example, subcutaneously, intravenously, intra-arterially, intraperitoneally, or otherwise. Generally, the time at which the lower titer of antibodies detected for the alternate routes of administration is detected should be roughly comparable; for example, the titer of antibodies can be determined at about 1 week, at about 2 weeks, at about 3 weeks, at about 4 weeks, at about 2 months, or at about 6 months following administration of the particle with the delivery construct or by injection.

Accordingly, in certain embodiments, the invention provides a method for delivering a particle to the bloodstream a subject that comprises contacting a delivery construct of the invention that comprises the particle to be delivered to an apical surface of a polarized epithelial cell of the subject, such that the particle is administered to the bloodstream of the subject, wherein a lower titer of antibodies specific for the particle is induced in the serum of the subject than is induced by subcutaneously administering the particle separately from the remainder of the delivery construct to a subject. In certain embodiments, the antibodies that are induced in the subject are specific for a biologically active agent delivered as part of the particle, wherein a lower titer of antibodies specific for the biologically active agent is induced in the serum of the subject than is induced by subcutaneously administering the biologically active agent in the absence of the particle. In certain embodiments, the antibodies that are induced in the subject are specific for a biologically active agent delivered as part of the particle, wherein a lower titer of antibodies specific for the biologically active agent is induced in the serum of the subject than is induced by subcutaneously administering particles comprising the biologically active agent.

In certain embodiments, the titer of antibodies specific for the particle induced in the serum of the subject by the particle delivered by the delivery construct is less than about 95% of the titer of antibodies induced by subcutaneously administering the particle separately from the remainder of the delivery construct. In certain embodiments, the titer of antibodies specific for the particle induced in the serum of the subject by the particle delivered by the delivery construct is less than about 90% of the titer of antibodies induced by subcutaneously administering the particle separately from the remainder of the delivery construct. In certain embodiments, the titer of antibodies specific for the particle induced in the serum of the subject by the particle delivered by the delivery construct is less than about 85% of the titer of antibodies induced by subcutaneously administering the particle separately from the remainder of the delivery construct. In certain embodiments, the titer of antibodies specific for the particle induced in the serum of the subject by the particle delivered by the delivery construct is less than about 80% of the titer of antibodies induced by subcutaneously administering the particle separately from the remainder of the delivery construct. In certain embodiments, the titer of antibodies specific for the particle induced in the serum of the subject by the particle delivered by the delivery construct is less than about 75% of the titer of antibodies induced by subcutaneously administering the particle separately from the remainder of the delivery construct.

In certain embodiments, the titer of antibodies specific for the particle induced in the serum of the subject by the particle delivered by the delivery construct is less than about 70% of the titer of antibodies induced by subcutaneously administering the particle separately from the remainder of the delivery construct. In certain embodiments, the titer of antibodies specific for the particle induced in the serum of the subject by the particle delivered by the delivery construct is less than about 65% of the titer of antibodies induced by subcutaneously administering the particle separately from the remainder of the delivery construct. In certain embodiments, the titer of antibodies specific for the particle induced in the serum of the subject by the particle delivered by the delivery construct is less than about 60% of the titer of antibodies induced by subcutaneously administering the particle separately from the remainder of the delivery construct. In certain embodiments, the titer of antibodies specific for the particle induced in the serum of the subject by the particle delivered by the delivery construct is less than about 55% of the titer of antibodies induced by subcutaneously administering the particle separately from the remainder of the delivery construct. In certain embodiments, the titer of antibodies specific for the particle induced in the serum of the subject by the particle delivered by the delivery construct is less than about 55% of the titer of antibodies induced by subcutaneously administering the particle separately from the remainder of the delivery construct.

In certain embodiments, the titer of antibodies specific for the particle induced in the serum of the subject by the particle delivered by the delivery construct is less than about 50% of the titer of antibodies induced by subcutaneously administering the particle separately from the remainder of the delivery construct. In certain embodiments, the titer of antibodies specific for the particle induced in the serum of the subject by the particle delivered by the delivery construct is less than about 45% of the titer of antibodies induced by subcutaneously administering the particle separately from the remainder of the delivery construct. In certain embodiments, the titer of antibodies specific for the particle induced in the serum of the subject by the particle delivered by the delivery construct is less than about 40% of the titer of antibodies induced by subcutaneously administering the particle separately from the remainder of the delivery construct. In certain embodiments, the titer of antibodies specific for the particle induced in the serum of the subject by the particle delivered by the delivery construct is less than about 35% of the titer of antibodies induced by subcutaneously administering the particle separately from the remainder of the delivery construct. In certain embodiments, the titer of antibodies specific for the particle induced in the serum of the subject by the particle delivered by the delivery construct is less than about 30% of the titer of antibodies induced by subcutaneously administering the particle separately from the remainder of the delivery construct.

In certain embodiments, the titer of antibodies specific for the particle induced in the serum of the subject by the particle delivered by the delivery construct is less than about 25% of the titer of antibodies induced by subcutaneously administering the particle separately from the remainder of the delivery construct. In certain embodiments, the titer of antibodies specific for the particle induced in the serum of the subject by the particle delivered by the delivery construct is less than 20% of the titer of antibodies induced by subcutaneously administering the particle separately from the remainder of the delivery construct. In certain embodiments, the titer of antibodies specific for the particle induced in the serum of the subject by the particle delivered by the delivery construct is less than about 15% of the titer of antibodies induced by subcutaneously administering the particle separately from the remainder of the delivery construct. In certain embodiments, the titer of antibodies specific for the particle induced in the serum of the subject by the particle delivered by the delivery construct is less than about 10% of the titer of antibodies induced by subcutaneously administering the particle separately from the remainder of the delivery construct. In certain embodiments, the titer of antibodies specific for the particle induced in the serum of the subject by the particle delivered by the delivery construct is less than about 5% of the titer of antibodies induced by subcutaneously administering the particle separately from the remainder of the delivery construct. In certain embodiments, the titer of antibodies specific for the particle induced in the serum of the subject by the particle delivered by the delivery construct is less than about 1% of the titer of antibodies induced by subcutaneously administering the particle separately from the remainder of the delivery construct.

### 6.3.1. Methods of Administration

The delivery constructs of the invention can be administered to a subject by any method known to one of skill in the art. In certain embodiments, the delivery constructs are contacted to a mucosal membrane of the subject. For example, the mucosal membrane can be present in the eye, nose, mouth, trachea, lungs, esophagus, stomach, small intestine, large intestine, rectum, anus, sweat glands, vulva, vagina, or penis of the subject. Preferably, the mucosal membrane is a mucosal membrane present in the digestive tract of the subject, such as a mucosal membrane in the mouth, esophagus, stomach, small intestine, large intestine, or rectum of the subject.

In such embodiments, the delivery constructs are preferably administered to the subject orally. Thus, the delivery construct can be formulated to protect the delivery construct from degradation in the acid environment of the stomach, if necessary. For example, many embodiments of the delivery constructs of the invention comprise polypeptide domains with defined activities. Unless such delivery constructs are protected from acid and/or enzymatic hydrolysis in the stomach, the constructs will generally be digested before delivery of substantial amounts of the particle to be delivered. Accordingly, composition formulations that protect the delivery construct from degradation can be used in administration of these delivery constructs. Examples of such compositions are provided below.

### 6.3.2. Dosage

Generally, a pharmaceutically effective amount of the delivery construct of the invention is administered to a subject. The skilled artisan can readily determine if the dosage of the delivery construct is sufficient to deliver an effective amount of the particle, as described below. In certain embodiments, between about 1 μg and about 1 g of delivery construct is administered. In other embodiments, between about 10 μg and about 500 mg of delivery construct is administered. In still other embodiments, between about 10 μg and about 100 mg of delivery construct is administered. In yet other embodiments, between about 10 μg and about 1000 μg of delivery construct is administered. In still other embodiments, between about 10 μg and about 250 μg of delivery construct is administered. In yet other embodiments, between about 10 μg and about 100 μg of delivery construct is administered. Preferably, between about 10 μg and about 50 μg of delivery construct is administered.

The volume of a composition comprising the delivery construct that is administered will generally depend on the concentration of delivery construct and the formulation of the composition. In certain embodiments, a unit dose of the delivery construct composition is between about 0.05 ml and about 1 ml, preferably about 0.5 ml. The delivery construct compositions can be prepared in dosage forms containing between 1 and 50 doses (e.g., 0.5 ml to 25 ml), more usually between 1 and 10 doses (e.g., 0.5 ml to 5 ml)

The delivery construct compositions of the invention can be administered in one dose or in multiple doses. A dose can be followed by one or more doses spaced by about 1 to about 6 hours, by about 6 to about 12 hours, by about 12 to about 24 hours, by about 1 day to about 3 days, by about 1 day to about 1 week, by about 1 week to about 2 weeks, by about 2 weeks to about 1 month, by about 4 to about 8 weeks, by about 1 to about 3 months, or by about 1 to about 6 months.

The particles to be delivered are generally particles for which a large amount of knowledge regarding dosage, frequency of administration, and methods for assessing effective concentrations in subjects has accumulated. Such knowledge can be used to assess efficiency of delivery, effective concentration of the particle in the subject, and frequency of administration. Thus, the knowledge of those skilled in the art can be used to determine whether, for example, the amount of particle delivered to the subject is an effective amount, the dosage should be increased or decreased, the subject should be administered the delivery construct more or less frequently, and the like.

### 6.3.3. Determining Amounts of Particle Delivered

The methods of the invention can be used to deliver, either locally or systemically, a pharmaceutically effective amount of a particle to a subject. The skilled artisan can determine whether the methods result in delivery of such a pharmaceutically effective amount of the particle. The exact methods will depend on the particle that is delivered, but generally will rely on either determining the concentration of the particle in the blood of the subject or in the biological compartment of the subject where the particle exerts its effects. Alternatively or additionally, the effects of the particle on the subject can be monitored. One exemplary method for determining the concentration of the particle in a fluid is by performing an ELISA assay, but any other suitable assay known to the skilled artisan can be used.

Any effect of a particle that is administered that is known by one of skill in the art, without limitation, can be assessed in determining whether an effective amount of the particle has been administered. Exemplary effects include, but are not limited to, receptor binding, receptor activation, downstream effects of receptor binding, downstream effects of receptor activation, coordination of compounds, effective blood clotting, bone growth, wound healing, cellular proliferation, contrasting in imaging, disease treatment, etc. The exact effect that is assessed will depend on the particle that is delivered.

### 6.4. Compositions Comprising Delivery Constructs

The delivery constructs of the invention can be formulated as compositions. The compositions are generally formulated appropriately for the immediate use intended for the delivery construct. For example, if the delivery construct is not to be administered immediately, the delivery construct can be formulated in a composition suitable for storage. One such composition is a lyophilized preparation of the delivery construct together with a suitable stabilizer. Alternatively, the delivery construct composition can be formulated for storage in a solution with one or more suitable stabilizers. Any such stabilizer known to one of skill in the art without limitation can be used. For example, stabilizers suitable for lyophilized preparations include, but are not limited to, sugars, salts, surfactants, proteins, chaotropic agents, lipids, and amino acids. Stabilizers suitable for liquid preparations include, but are not limited to, sugars, salts, surfactants, proteins, chaotropic agents, lipids, and amino acids. Specific stabilizers than can be used in the compositions include, but are not limited to, trehalose, serum albumin, phosphatidylcholine, lecithin, and arginine. Other compounds, compositions, and methods for stabilizing a lyophilized or liquid preparation of the delivery constructs may be found, for example, in U.S. Pat. Nos. 6,573,237, 6,525,102, 6,391,296, 6,255,284, 6,133,229, 6,007,791, 5,997,856, and 5,917,021.

Further, the delivery construct compositions of the invention can be formulated for administration to a subject. Such compositions generally comprise one or more delivery constructs of the invention and a pharmaceutically acceptable excipient, diluent, carrier, or vehicle. Any such pharmaceutically acceptable excipient, diluent, carrier, or vehicle known to one of skill in the art without limitation can be used. Examples of a suitable excipient, diluent, carrier, or vehicle can be found in Remington's Pharmaceutical Sciences, 21st Ed. 2005, Mack Publishing Co., Easton.

In certain embodiments, the delivery construct compositions are formulated for nasal administration.

In certain embodiments, the delivery construct compositions are formulated for oral administration. In such embodiments, the compositions can be formulated to protect the delivery construct from acid and/or enzymatic degradation in the stomach. Upon passage to the neutral to alkaline environment of the duodenum, the delivery construct then contacts a mucous membrane and is transported across the polarized epithelial membrane. The delivery constructs may be formulated in such compositions by any method known by one of skill in the art, without limitation.

In certain embodiments, the oral formulation comprises a delivery construct and one or more compounds that can protect the delivery construct while it is in the stomach. For example, the protective compound should be able to prevent acid and/or enzymatic hydrolysis of the delivery construct. In certain embodiments, the oral formulation comprises a delivery construct and one or more compounds that can facilitate transit of the construct from the stomach to the small intestine. In certain embodiments, the one or more compounds that can protect the delivery construct from degradation in the stomach can also facilitate transit of the construct from the stomach to the small intestine. Preferably, the oral formulation comprises one or more compounds that can protect the delivery construct from degradation in the stomach and facilitate transit of the construct from the stomach to the small intestine. For example, inclusion of sodium bicarbonate can be useful in facilitating the rapid movement of intra-gastric delivered materials from the stomach to the duodenum as described in Mrsny et al., 1999, Vaccine 17:1425-1433.

Other methods for formulating compositions so that the delivery constructs can pass through the stomach and contact polarized epithelial membranes in the small intestine include, but are not limited to, enteric-coating technologies as described in DeYoung, 1989, Int J Pancreatol. 5 Suppl:31-6, and the methods provided in U.S. Pat. Nos. 6,613,332, 6,174,529, 6,086,918, 5,922,680, and 5,807,832.

### 6.4.1. Kits Comprising Compositions

In yet another aspect, the invention provides a kit that comprises a composition of the invention. In certain embodiments, the kit further comprises instructions that direct administration of the composition to a mucous membrane of the subject to whom the composition is administered. In certain embodiments, the kit further comprises instructions that direct oral administration of the composition to the subject to whom the composition is administered.

In certain embodiments, the kit comprises a composition of the invention in more or more containers. In certain embodiments, the composition can be in a unit dosage form, e.g., a tablet, lozenge, capsule, etc. In certain embodiments, the composition can be provided in or with a device for administering the composition, such as, for example, a device configured to administer a single-unit dose of the composition, e.g., an inhaler.

### 6.5. Making and Testing Delivery Constructs

The delivery constructs of the invention are preferably produced recombinantly, as described below. However, the delivery constructs may also be produced by chemical synthesis using methods known to those of skill in the art.

### 6.5.1. Manufacture of Delivery Constructs

Methods for expressing and purifying the delivery constructs of the invention are described extensively in the examples below. Generally, the methods rely on introduction of an expression vector encoding the receptor binding domain and the translocation domain and, optionally, the cleavable linker, of the delivery construct to a cell that can express this portion of the delivery construct from the vector. This portion of the delivery construct can then be connected with the particle using any appropriate technique known by one skilled in the art. The delivery construct can then be purified for administration to a subject.

### 6.5.2. Testing Delivery Constructs

Having selected the domains of the delivery construct, the function of these domains, and of the delivery constructs as a whole, can be routinely tested to ensure that the constructs can deliver a particle across mucous membranes of a subject free from the remainder of the construct. For example, the delivery constructs can be tested for cell recognition, transcytosis and cleavage using routine assays. The entire chimeric protein can be tested, or, the function of various domains can be tested by substituting them for native domains of the wild-type toxin.

### 6.5.2.1. Receptor Binding/Cell recognition

Receptor binding domain function can be tested by monitoring the delivery construct's ability to bind to the target receptor. Such testing can be accomplished using cell-based assays, with the target receptor present on a cell surface, or in cell-free assays. For example, delivery construct binding to a target can be assessed with affinity chromatography. The construct can be attached to a matrix in an affinity column, and binding of the receptor to the matrix detected, or vice versa. Alternatively, if antibodies have been identified that bind to either the receptor binding domain or its cognate receptor, the antibodies can be used, for example, to detect the receptor binding domain in the delivery construct by immunoassay, or in a competition assay for the cognate receptor. An exemplary cell-based assay that detects delivery construct binding to receptors on cells comprises labeling the construct and detecting its binding to cells by, e.g., fluorescent cell sorting, autoradiography, etc.

### 6.5.2.2. Transcytosis

The function of the transcytosis domain can be tested as a function of the delivery construct's ability to pass through an epithelial membrane. Because transcytosis first requires binding to the cell, these assays can also be used to assess the function of the cell recognition domain.

The delivery construct's transcytosis activity can be tested by any method known by one of skill in the art, without limitation. In certain embodiments, transcytosis activity can be tested by assessing the ability of a delivery construct to enter a non-polarized cell to which it binds. Without intending to be bound to any particular theory or mechanism of action, it is believed that the same property that allows a transcytosis domain to pass through a polarized epithelial cell also allows molecules bearing the transcytosis domain to enter non-polarized cells. Thus, the delivery construct's ability to enter the cell can be assessed, for example, by detecting the physical presence of the construct in the interior of the cell. For example, the delivery construct can be labeled with, for example, a fluorescent marker, and the delivery construct exposed to the cell. Then, the cells can be washed, removing any delivery construct that has not entered the cell, and the amount of marker remaining determined. Detecting the marker in this cellular fraction indicates that the delivery construct has entered the cell.

In other embodiments, the delivery construct's transcytosis ability can be tested by assessing the delivery construct's ability to pass through a polarized epithelial cell. For example, the delivery construct can be labeled with, for example, a fluorescent marker and contacted to the apical membranes of a layer of epithelial cells. Fluorescence detected on the basal-lateral side of the membrane formed by the epithelial cells indicates that the transcytosis domain is functioning properly.

### 6.5.2.3. Cleavable Linker Cleavage

The function of the optional cleavable linker can generally be tested in a cleavage assay. Any suitable cleavage assay known by one of skill in the art, without limitation, can be used to test the cleavable linkers. Both cell-based and cell-free assays can be used to test the ability of an enzyme to cleave the cleavable linkers.

An exemplary cell-free assay for testing cleavage of cleavable linkers comprises preparing extracts of polarized epithelial cells and exposing a labeled delivery construct bearing a cleavable linker to the fraction of the extract that corresponds to membrane-associated enzymes. In such assays, the label can be attached to either the particle to be delivered or to the remainder of the delivery construct. Among these enzymes are cleavage enzymes found near the basal-lateral membrane of a polarized epithelial cell, as described above. Cleavage can be detected, for example, by binding the delivery construct with, for example, an antibody and washing off unbound molecules. If label is attached to the particle to be delivered, then little or no label should be observed on the molecule bound to the antibodies. Alternatively, the binding agent used in the assay can be specific for the particle, and the remainder of the construct can be labeled. In either case, cleavage can be assessed.

Cleavage can also be tested using cell-based assays that test cleavage by polarized epithelial cells assembled into membranes. For example, a labeled delivery construct, or portion of a delivery construct comprising the cleavable linker, can be contacted to either the apical or basolateral side of a monolayer of suitable epithelial cells, such as, for example, Caco-2 cells, under conditions that permit cleavage of the linker. Cleavage can be detected by detecting the presence or absence of the label using a reagent that specifically binds the delivery construct, or portion thereof. For example, an antibody specific for the delivery construct can be used to bind a delivery construct comprising a label distal to the cleavable linker in relation to the portion of the delivery construct bound by the antibody. Cleavage can then be assessed by detecting the presence of the label on molecules bound to the antibody. If cleavage has occurred, little or no label should be observed on the molecules bound to the antibody. By performing such experiments, enzymes that preferentially cleave at the basolateral membrane rather than the apical membrane can be identified, and, further, the ability of such enzymes to cleave the cleavable linker in a delivery construct can be confirmed.

Further, cleavage can also be tested using a fluorescence reporter assay as described in U.S. Pat. No. 6,759,207. Briefly, in such assays, the fluorescence reporter is contacted to the basolateral side of a monolayer of suitable epithelial cells under conditions that allow the cleaving enzyme to cleave the reporter. Cleavage of the reporter changes the structure of the fluorescence reporter, changing it from a non-fluorescent configuration to a fluorescent configuration. The amount of fluorescence observed indicates the activity of the cleaving enzyme present at the basolateral membrane.

Further, cleavage can also be tested using an intra-molecularly quenched molecular probe, such as those described in U.S. Pat. No. 6,592,847. Such probes generally comprise a fluorescent moiety that emits photons when excited with light of appropriate wavelength and a quencher moiety that absorbs such photons when in close proximity to the fluorescent moiety. Cleavage of the probe separates the quenching moiety from the fluorescent moiety, such that fluorescence can be detected, thereby indicating that cleavage has occurred. Thus, such probes can be used to identify and assess cleavage by particular cleaving enzymes by contacting the basolateral side of a monolayer of suitable epithelial cells with the probe under conditions that allow the cleaving enzyme to cleave the probe. The amount of fluorescence observed indicates the activity of the cleaving enzyme being tested.

### 7. EXAMPLES

The following examples merely illustrate the invention, and are not intended to limit the invention in any way.

7.1. Construction of a Delivery Construct

This example describes construction of two plasmids for expressing the recepter binding domain and translocation domains of an exemplary particle delivery construct. Using conventional techniques, one construct was prepared encoding a mutant form of Pseudomonas aeruginosa exotoxin A (PE) made non-toxic by deletion of a glutamic acid at position 553 (ΔE553PE or ntPE) and having green fluorescent protein (GFP) positioned at its C-terminus. Another ntPE-GFP plasmid construct was similarly prepared that would have reduced affinity for the cell surface receptor CD91 by substitution of arginine at position 57 with glutamic acid (K57 ntPE-GFP).

The proteins were expressed in E. coli DH5α cells (Invitrogen, Carlsbad, Calif.) following transformation with ntPE-GFP or K57 ntPE-GFP plasmids by heat-shock (1 min at 42° C.). Transformed cells, selected on antibiotic-containing media, were isolated and grown in Luria-Bertani broth (Difco). Protein expression was induced by addition of 1 mM isopropyl-D-thiogalactopyranoside (IPTG). Two hours following IPTG induction, cells were harvested by centrifugation at 5,000×g for 10 min at 4° C. Inclusion bodies were isolated following cell lysis and proteins were solubilized in 6 M guanidine HCl and 2 mM EDTA (pH 8.0) plus 65 mM dithiothreitol. Following refolding and purification as previously described in Hertle et al., 2001, Infect. Immun. 69:6962-69, proteins were stored at 5 mg/ml in PBS (pH 7.4) lacking Ca²⁺ and Mg²⁺ at −80° C. Proper folding of ntPE-GFP and K57 ntPE-GFP was assessed by acquisition and retention of the fluorescence signature associated with this fluorescent protein. Reference green fluorescent protein (GFP) for comparison was purchased from Upstate (Charlottesville, Va.).

7.2. Characterization of a Delivery Construct

The following procedures can be used to assess proper refolding of a delivery construct. The protein refolding process is monitored by measuring, e.g., binding activity of an exmplary Delivery Construct with ntPE binding receptor and CD 91 receptors on a Biacore SPR instrument (Biacore, Sweden) according to the manufacturer's instructions. Proper refolding of delivery constructs and particles comprising elements in need of such refolding in exemplary constructs can be tested in similar binding assays with appropriate binding agents. By testing such binding affinities, the skilled artisan can assess the proper folding of each portion of the delivery construct.

7.3. Particle Coating

This example describes the association of the receptor binding domain and translocation domain of an exemplary delivery construct with an exemplary particle to be delivered. It should be noted that this exemplary delivery construct does not comprise a cleavable linker; however, the presence or absence of such cleavable linker is not expected to affect binding and/or transcytosis of the delivery construct.

Polystyrene beads (100 nm in diameter) containing a covalently integrated red fluorescent dye with excitation/emission properties of 468/508 nm and having aldehyde surface functional groups (XPR-582) were purchased from Duke Scientific (Palo Alto, Calif.). The beads, as used in this example, represent an exemplary particle. One hundred μl of XPR-582 beads (at 2% solids) was mixed with approximately 2.5 nmoles GFP, ntPE-GFP or K57 ntPE-GFP (prepared as described above) in a final volume of 200 μl neutral (pH 7.0) phosphate buffered saline (PBS). After 2 hr of gentle rocking at room temperature 20 μl of 2 mg/ml bovine serum albumin (BSA; Sigma, St. Louis, Mo.) solution in PBS was added. Preparations were then dialyzed by three cycles of dilution with PBS and concentrated using a 100,000 molecular weight cutoff Microcon filter device from Millipore (Bedford, Mass.). Final preparations of coated beads were at 1% solids.

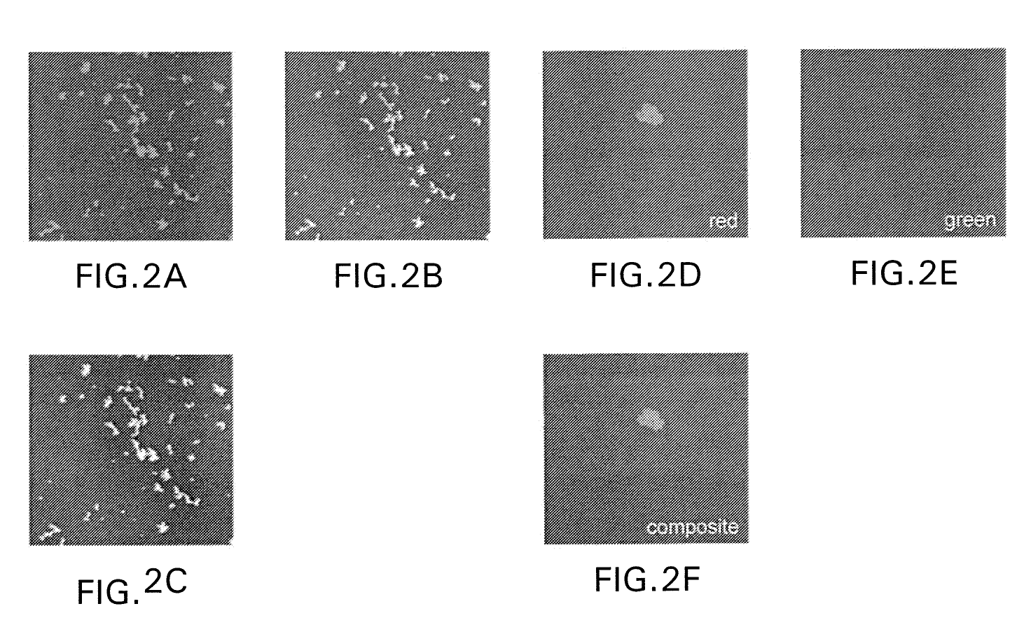

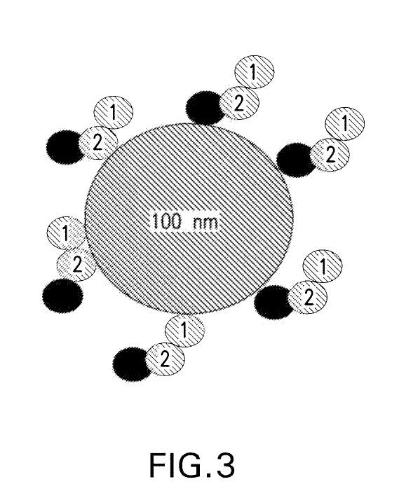

The presence of ntPE-GFP coupling to the particles was verified by co-localization of GFP and inherent particle fluorescence energies using confocal microscopy. See FIGS. 2A-C. Dual signal fluorescence was verified as specific for the coated GFP-containing protein by similar analysis of particles prepared similarly where bovine serum albumin was used to couple surface accessible aldehyde residues. See FIGS. 2D-F. For improved visual clarity of this fluorescent microscopic analysis, aggregates of particles are presented in FIGS. 2A-F. Although these co-localizations could verify that particles were coated with the desired material, they did not address the organization of the protein at the particle surface. Thus, a variety of potential conjugation outcomes could have occurred, as shown in FIG. 3. Nonetheless, a sufficient number of conjugation events that permit effective transcytosis of the particle occurred as shown by Example 4, below. Thus, the exact arrangement of the protein at the particle surface was not critical to proper function of the delivery construct.

7.4. Delivery of an Exemplary Particle

This example describes transcytosis of an exemplary delivery construct across a polarized epithelial cell monolayer. Caco-2 cells were grown to confluent monolayers on collagen-coated 0.4-μm pore size polycarbonate membrane transwell supports (Corning-Costar, Cambridge, Mass.) and used 18-25 days after attainment of trans-epithelial electrical resistance of >250Ω·cm2 as measured using a chopstick Millicell-ERS® voltmeter (Millipore). Particles coated with ntPE-GFP, K57 ntPE-GFP, or GFP (diluted 1:10 in culture media) prepared according to Example 6.3 were added to the apical surface of confluent monolayers that were then returned to the cell culture incubator.

After 6 or 24 hrs, monolayers were washed with Caco-2 cell culture media, fixed in absolute ethanol (20 minutes, −20° C.), and blocked for 1 hr at room temperature in 5% normal goat serum. After incubation in a humidity chamber for 1 hour with primary antibodies to JAM-A (C. A. Parkos, Emory University, Atlanta, Ga.), cell filters were washed, probed with Alexa 488-conjugated goat anti-mouse/-rabbit IgG (1 hr, RT; Molecular Probes, Eugene, Oreg.) and mounted on slides with anti-fade reagent prior to visualization on an LSM510 confocal microscope (Zeiss Microimaging, Thornwood, N.Y.). Images shown are representative of at least three experiments, with multiple images taken per slide.

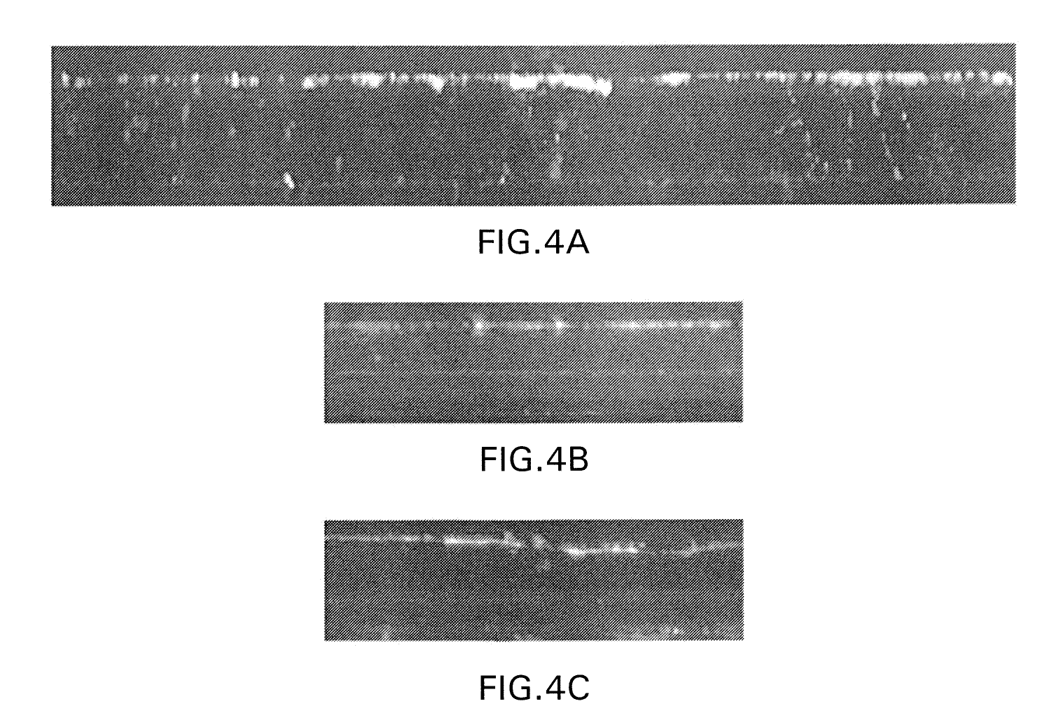

Particles coated with ntPE-GFP were found to transport across confluent monolayers of polarized Caco-2 cells in vitro by 6 hr following apical application. See FIG. 4A. At this same time particles coated with K57 ntPE-GFP (FIG. 4B) or GFP (FIG. 4C) alone did not demonstrate significant uptake into or transport across Caco-2 monolayers following apical application to the cells. Occasionally, aggregates of ntPE-GFP particles could be observed in association with the apical surface as well as internalized by Caco-2 cells. See FIG. 4A. Similar sporadic associations were observed with particle aggregates present in the K57 ntPE-GFP and GFP preparations at 6 hr post apical application. See FIGS. 4B and 4C. In general, these aggregates remained at or near the apical surface of monolayers and co-localized with an antibody used to mark the cellular distribution of ICAM/JAM-c at the plasma membrane. See FIGS. 4B and 4C.

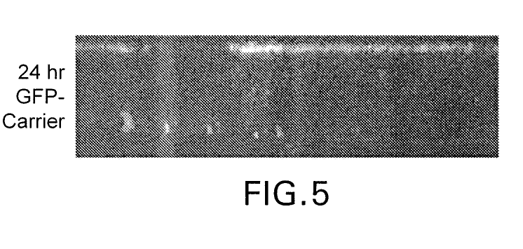

Examination of monolayers after 24 hr of apical exposure of particle preparations further supported the uptake and transport potential of ntPE. See FIG. 5. Accordingly, these experiments demonstrate that ntPE, conjugated to an exemplary particle of 100 nm diameter, functions to transport the particles across polarized epithelial cells following application to the apical surface of such cells.

7.5. Delivery of an Exemplary Particle in an In Vivo System

This example describes delivery of a particle to an exemplary model organism with an exemplary delivery construct. In this example, the exemplary particle delivered is aggregated insulin.

First, 100 units of regular insulin (Novo Nordisk) in 2 mls buffer was adjusted to pH 5.0 with MES buffer and zinc chloride were added to a final concentration of 1 mM. The insulin was then incubated at room temperature for 10 minutes to allow the insulin molecules to aggregate.

Next, either 2 mg (1×) or 4 mg (2×) ntPE was added to 50 Units aggregated insulin to test the effects of different ratios of polypeptide to particle. 100 mg ethylene diimine carbodiimide was then added to the reaction mixture to cross-link the insulin aggregates and nt-PE, then the reaction was incubated on ice for 30 minutes. The delivery constructs thus made were then dialyzed overnight against pH 7 phosphate-buffered saline.

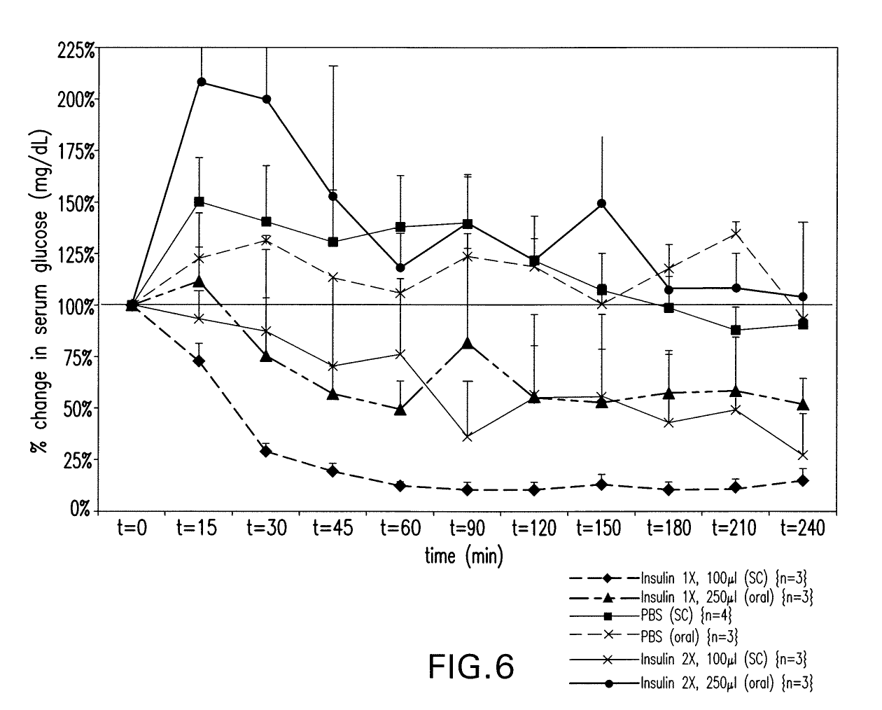

To assess the activity of the delivery constructs, either 100 μl by subcutaneous injection or 250 μl by oral gavage of the 1× delivery construct, the 2× delivery construct, or PBS as negative control was administered to fasted female STZ BALB/c mice. Serum blood glucose was monitored every 15 minutes for the first hour, then every 30 minutes thereafter, to assess the effects of the insulin aggregates delivered with the delivery constructs. Experiments were performed in triplicate and results are presented as an average of the three experiments. The results of the experiment are presented as FIG. 6.

As shown in FIG. 6, the 1× delivery construct administered subcutaneously resulted in the greatest decrease in blood glucose concentration. Similarly, oral administration of the 1× delivery construct also resulted in a substantial decrease in blood glucose concentration. Thus, the 1× delivery construct effectively delivered the aggregated insulin in a bioactive form to the tested animals. The 2× delivery construct did not work as well as the 1× delivery construct, suggesting that routine optimization of the ratio of polypeptide carrier to particle can increase or optimize the efficiency of particle delivery. Finally, the PBS negative control demonstrates that the stress of oral gavage (and, to a lesser extent, subcutaneous injection) of mice results in release of glucose from energy reserves. Thus, the increased glucose concentrations observed following oral administration of the 2× delivery construct can be attributed to this effect. It should be noted that the increase observed from oral administration of the 2× delivery construct was less than that observed for the appropriate negative control, suggesting that the 2× delivery construct was also able to deliver bioactive insulin aggregates to test animals. Thus, these results demonstrate that the delivery constructs of the invention can be used to deliver a particle comprising an aggregate of a bioactive molecule to the serum of a representative test animal and that such aggregates can exert a biological effect in the animal once delivered.

The present invention provides, inter alia, delivery constructs and methods of delivering a particle to a subject. While many specific examples have been provided, the above description is intended to illustrate rather than limit the invention. Many variations of the invention will become apparent to those skilled in the art upon review of this specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. Citation of these documents is not an admission that any particular reference is “prior art” to this invention.

What is claimed is:

1. A delivery construct, comprising:
a)—a receptor binding domain,
b)—a transcytosis domain, and
c)—a particle.

2. The delivery construct of claim 1, wherein the particle is a metal particle, a liposphere, a porous particle, a cell, a peptide or polypeptide aggregate, a peptide or polypeptide crystal, or a high-contrast.

3. The delivery construct of claim 1, wherein the particle is a platinum or gold particle.

4. The delivery construct of claim 1, wherein the particle is a liposphere.

5. The delivery construct of claim 1, wherein the particle is a porous particle.

6. The delivery construct of claim 1, wherein the particle is a cell.

7. The delivery construct of claim 6, wherein the cell is a mammalian cell.

8. The delivery construct of claim 6, wherein the cell is a human, rat, mouse, dog, hamster, chicken, or monkey cell.

9. The delivery construct of claim 1, wherein the particle is a high-contrast particle.

10. The delivery construct of claim 1, wherein the particle is a peptide or polypeptide aggregate.

11. The delivery construct of claim 1, wherein the particle is a peptide or polypeptide crystal.

12. The delivery construct of claim 1, further comprising a cleavable linker, wherein cleavage at the cleavable linker separates the particle from the remainder of the delivery construct.

13. The delivery construct of claim 12, further comprising a second cleavable linker.

14. The delivery construct of claim 12, wherein the cleavable linker comprises an amino acid sequence that is selected from the group consisting of Ala-Ala-Pro-Phe (SEQ ID NO.:4), Gly-Gly-Phe (SEQ ID NO.:5), Ala-Ala-Pro-Val (SEQ ID NO.:6), Gly-Gly-Leu (SEQ ID NO.:7), Ala-Ala-Leu (SEQ ID NO.:8), Phe-Val-Arg (SEQ ID NO.:9), Val-Gly-Arg (SEQ ID NO.: 10).

15. The delivery construct of claim 12, wherein the cleavable linker is cleavable with an enzyme selected from the group consisting of Cathepsin GI, Chymotrypsin I, Elastase I, Subtilisin AI, Subtilisin AII, Thrombin I, and Urokinase I.

16. The delivery construct of claim 1, wherein the receptor binding domain is selected from the group consisting of receptor binding domains from Pseudomonas exotoxin A, cholera toxin, botulinum toxin, diphtheria toxin, shiga toxin, or shiga-like toxin; monoclonal antibodies; polyclonal antibodies; single-chain antibodies; TGF α; EGF; IGF-I; IGF-II; IGF-III; IL-1; IL-2; IL-3; IL-6; MIP-1a; MIP-1b; MCAF; and IL-8.

17. The delivery construct of claim 1, wherein the receptor binding domain binds to a cell-surface receptor that is selected from the group consisting of α2-macroglobulin receptor, epidermal growth factor receptor, transferrin receptor, chemokine receptor, CD25, CD11B, CD11C, CD80, CD86, TNFα receptor, TOLL receptor, M-CSF receptor, GM-CSF receptor, scavenger receptor, and VEGF receptor.

18. The delivery construct of claim 16, wherein the receptor binding domain of Pseudomonas exotoxin A is Domain Ia of Pseudomonas exotoxin A.

19. The delivery construct of claim 18, wherein the receptor binding domain of Pseudomonas exotoxin A has an amino acid sequence that is SEQ ID NO.:1.

20. The delivery construct of claim 1, wherein the transcytosis domain is selected from the group consisting of transcytosis domains from Pseudomonas exotoxin A, botulinum toxin, diphtheria toxin, pertussis toxin, cholera toxin, heat-labile E. coli enterotoxin, shiga toxin, and shiga-like toxin.

21. The delivery construct of claim 20, wherein the transcytosis domain is Pseudomonas exotoxin A transcytosis domain.

22. The delivery construct of claim 21, wherein the Pseudomonas exotoxin A transcytosis domain has an amino acid sequence that is SEQ ID NO.:2.

23. A composition comprising a delivery construct, the delivery construct comprising:
a)—a receptor binding domain,
b)—a transcytosis domain, and
c)—a particle.

24. The composition of claim 23, wherein the composition further comprises a pharmaceutically acceptable diluent, excipient, vehicle, or carrier.

25. The composition of claim 23, wherein the composition is formulated for nasal or oral administration.

26. A method for delivering a particle to a subject, comprising contacting an apical surface of a polarized epithelial cell of the subject with a delivery construct, wherein the delivery construct comprises a receptor binding domain, a transcytosis domain, and the particle, wherein the transcytosis domain transcytosis the macromolecule to and through the basal-lateral membrane of the epithelial cell.

27. The method of claim 26, wherein the particle is a metal particle, a liposphere, a porous particle, a cell, or a high-contrast particle.

28. The method of claim 26, wherein the particle is a platinum or gold particle.

29. The method of claim 26, wherein the particle is a liposphere.

30. The method of claim 26, wherein the particle is a porous particle.

31. The method of claim 26, wherein the particle is a cell.

32. The method of claim 26, wherein the cell is a mammalian cell.

33. The method of claim 26, wherein the cell is a human, rat, mouse, dog, hamster, chicken, or monkey cell.

34. The method of claim 26, wherein the particle is a high-contrast particle.

35. The method of claim 26, wherein the particle is a peptide or polypeptide aggregate.

36. The method of claim 26, wherein the particle is a peptide or polypeptide crystal.

37. The method of claim 26, further comprising a cleavable linker, wherein cleavage at the cleavable linker separates the particle from the remainder of the delivery construct.

38. The method of claim 26, further comprising a second cleavable linker.

39. The method of claim 26, wherein the cleavable linker comprises an amino acid sequence that is selected from the group consisting of Ala-Ala-Pro-Phe (SEQ ID NO.:4), Gly-Gly-Phe (SEQ ID NO.:5), Ala-Ala-Pro-Val (SEQ ID NO.:6), Gly-Gly-Leu (SEQ ID NO.:7), Ala-Ala-Leu (SEQ ID NO.:8), Phe-Val-Arg (SEQ ID NO.:9), Val-Gly-Arg (SEQ ID NO.: 10).

40. The method of claim 26, wherein the cleavable linker is cleavable with an enzyme selected from the group consisting of Cathepsin GI, Chymotrypsin I, Elastase I, Subtilisin AI, Subtilisin AII, Thrombin I, and Urokinase I.

41. The method of claim 26, wherein the receptor binding domain is selected from the group consisting of receptor binding domains from Pseudomonas exotoxin A, cholera toxin, botulinum toxin, diphtheria toxin, shiga toxin, or shiga-like toxin; monoclonal antibodies; polyclonal antibodies; single-chain antibodies; TGF α; EGF; IGF-I; IGF-II; IGF-III; IL-1; IL-2; IL-3; IL-6; MIP-1a; MIP-1b; MCAF; and IL-8.

42. The method of claim 26, wherein the receptor binding domain binds to a cell-surface receptor that is selected from the group consisting of α2-macroglobulin receptor, epidermal growth factor receptor, transferrin receptor, chemokine receptor, CD25, CD11B, CD11C, CD80, CD86, TNFα receptor, TOLL receptor, M-CSF receptor, GM-CSF receptor, scavenger receptor, and VEGF receptor.

43. The method of claim 26, wherein the receptor binding domain of Pseudomonas exotoxin A is Domain Ia of Pseudomonas exotoxin A.

44. The method of claim 26, wherein the receptor binding domain of Pseudomonas exotoxin A has an amino acid sequence that is SEQ ID NO.:1.

45. The method of claim 26, wherein the transcytosis domain is selected from the group consisting of transcytosis domains from Pseudomonas exotoxin A, botulinum toxin, diphtheria toxin, pertussis toxin, cholera toxin, heat-labile E. coli enterotoxin, shiga toxin, and shiga-like toxin.

46. The method of claim 26, wherein the transcytosis domain is Pseudomonas exotoxin A transcytosis domain.

47. The method of claim 26, wherein the Pseudomonas exotoxin A transcytosis domain has an amino acid sequence that is SEQ ID NO.:2.

48. The method of claim 26, wherein the receptor binding domain is selected from the group consisting of receptor binding domains from Pseudomonas exotoxin A, cholera toxin, diphtheria toxin, shiga toxin, or shiga-like toxin; monoclonal antibodies; polyclonal antibodies; single-chain antibodies; TGF α; EGF; IGF-I; IGF-II; IGF-III; IL-1; IL-2; IL-3; IL-6; MIP-1a; MIP-1b; MCAF; and IL-8.

49. The method of claim 26, wherein the receptor binding domain binds to a cell surface receptor selected from the group consisting of α2-macroglobulin receptor, EGFR, IGFR, transferrin receptor, chemokine receptor, CD25, CD11B, CD11C, CD80, CD86, TNFα receptor, TOLL receptor, M-CSF receptor, GM-CSF receptor, scavenger receptor, and VEGF receptor.

50. The method of claim 26, wherein the transcytosis domain is selected from the group consisting of transcytosis domains from Pseudomonas exotoxin A, botulinum toxin, diphtheria toxin, pertussis toxin, cholera toxin, heat-labile E. coli enterotoxin, shiga toxin, and shiga-like toxin.

51. The method of claim 26, wherein the macromolecule is selected from the group consisting of a peptide, a polypeptide, a protein, a nucleic acid, and a lipid.

52. The method of claim 26, wherein the enzyme that is present at a basal-lateral membrane of a polarized epithelial cell is selected from the group consisting of Cathepsin GI, Chymotrypsin I, Elastase I, Subtilisin AI, Subtilisin AII, Thrombin I, and Urokinase I.

53. The method of claim 26, wherein the cleavable linker comprises an amino acid sequence that is selected from the group consisting of Ala-Ala-Pro-Phe (SEQ ID NO.:4), Gly-Gly-Phe (SEQ ID NO.:5), Ala-Ala-Pro-Val (SEQ ID NO.:6), Gly-Gly-Leu (SEQ ID NO.:7), Ala-Ala-Leu (SEQ ID NO.:8), Phe-Val-Arg (SEQ ID NO.:9), Val-Gly-Arg (SEQ ID NO.: 10).

54. The method of claim 26, wherein the epithelial cell is selected from the group consisting of nasal epithelial cells, oral epithelial cells, intestinal epithelial cells, rectal epithelial cells, vaginal epithelial cells, and pulmonary epithelial cells.

55. The method of claim 26, wherein the mammal is a human.

56. The method of claim 26, wherein the delivery construct contacts the apical membrane of the epithelial cell.


SEQUENCE LISTING (23 sequences)

SEQ ID NO: 1 - protein, 638 aa, Pseudomonas Aeruginosa
MHLIPHWIPLVASLGLLAGGSSASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQTQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRQWEQLEQCGYPVQRLVALYLAARLSWNQVDQVIRNALASPGSGGDLGEAIREQPEQARLALTLAAAESERFVRGGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 2 - protein, 154 aa, Pseudomonas Aeruginosa
TVISHRLHFPEGGSLAALTAHQACHLPLETFTRHRQPRQWEQLEQCGYPVQRLVALYLAARLSWNQVDQVIRNALASPGSGGDLGEAIREQPEQARLALTLAAAESERFVRGGTGNDEAGAANADVVSLTCPVAAGECAGPADSGDALLERNYP

SEQ ID NO: 3 - protein, 241 aa, Pseudomonas Aeruginosa
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQTQPRREKRWSEWASGKVLCLLDPLDGVYNYLAQQRCNLDDTWEGKIYRVLAGNPAKHDLDIKP

SEQ ID NO: 4 - protein, 4 aa, Artificial Sequence
  Protease cleavange recognition cite
AAPF

SEQ ID NO: 5 - protein, 4 aa, Artificial Sequence
  Protease cleavange recognition cite
GGFX

SEQ ID NO: 6 - protein, 4 aa, Artificial Sequence
  Protease cleavange recognition cite
AAPV

SEQ ID NO: 7 - protein, 4 aa, Artificial Sequence
  Protease cleavange recognition cite
GGLX

SEQ ID NO: 8 - protein, 4 aa, Artificial Sequence
  Protease cleavange recognition cite
AALX

SEQ ID NO: 9 - protein, 4 aa, Artificial Sequence
  Protease cleavange recognition cite
FVRX

SEQ ID NO: 10 - protein, 4 aa, Artificial Sequence
  Protease cleavange recognition cite
VGRX

SEQ ID NO: 11 - protein, 5 aa, Artificial Sequence
  Protease cleavange recognition cite
YVADX

SEQ ID NO: 12 - protein, 5 aa, Artificial Sequence
  Protease cleavange recognition cite
DXXDX

SEQ ID NO: 13 - protein, 9 aa, Artificial Sequence
  Protease cleavange recognition cite
RXXXXXXRX

SEQ ID NO: 14 - protein, 9 aa, Artificial Sequence
  Protease cleavange recognition cite
KXXXXXXRX

SEQ ID NO: 15 - protein, 6 aa, Artificial Sequence
  Protease cleavange recognition cite
GRTKRX

SEQ ID NO: 16 - protein, 5 aa, Artificial Sequence
  Protease cleavange recognition cite
RVRRX

SEQ ID NO: 17 - protein, 6 aa, Artificial Sequence
  Protease cleavange recognition cite
NRVRRX

SEQ ID NO: 18 - protein, 6 aa, Artificial Sequence
  Protease cleavange recognition cite
PXWVPX

SEQ ID NO: 19 - protein, 4 aa, Artificial Sequence
  Protease cleavange recognition cite
WVAX

SEQ ID NO: 20 - protein, 4 aa, Artificial Sequence
  Protease cleavange recognition cite
XFXX

SEQ ID NO: 21 - protein, 4 aa, Artificial Sequence
  Protease cleavange recognition cite
XYXX

SEQ ID NO: 22 - protein, 4 aa, Artificial Sequence
  Protease cleavange recognition cite
XWXX

SEQ ID NO: 23 - protein, 12 aa, Artificial Sequence
  Protease cleavange recognition cite
DRYIPFHLLXYX